(19) United States
(12) Patent Application Publication
(10) Pub. No.: US 2003/0162706 A1
(43) Pub. Date: Aug. 28, 2003

(54) Angiogenesis modulating proteins

(75) Inventors: Kevin Gene Peters, Loveland, OH (US); Larry Joseph Thompson, Anderson, IN (US); Feng Wang, Cincinnati, OH (US); Kenneth Donald Greis, Fort Thomas, KY (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/316,253

(22) Filed: Dec. 10, 2002

Related U.S. Application Data
(60) Provisional application No. 60/355,295, filed on Feb. 8, 2002.
(60) Provisional application No. 60/391,758, filed on Jun. 26, 2002.

(51) Int. Cl. A61K 39/395; A61K 38/17

(52) U.S. Cl. 514/12

(57) ABSTRACT

Angiogenesis modulating proteins may be useful for treating angiogenesis mediated disorders. Further, these angiogenesis modulating proteins may be useful to screen agents that are, in turn, useful for treating angiogenesis mediated disorders.

### CROSS REFERENCE TO RELATED APPLICATIONS

This application is related to U.S. Provisional Applications No. 60/355,295 and 60/391,758, filed Feb. 8, 2002 and Jun. 26, 2002, respectively, which are herein incorporated by reference in their entirety.

### FIELD OF INVENTION

This invention is directed to proteins that are useful in methods for modulating angiogenesis.

### BACKGROUND OF THE INVENTION

Angiogenesis, the sprouting of new blood vessels from the pre-existing vasculature, plays a crucial role in a wide range of physiological and pathological processes in Nguyen, L. L. et al, Int. Rev. Cytol., 204, 1-48, (2001). It is a complex process that is mediated by communication between the endothelial cells that line blood vessels and their surrounding environment, Glienke, J. et al, Eur. J. Biochem., 267, 2820-2830, (2000). In the early stages of angiogenesis, tissue or tumor cells produce and secrete pro-angiogenic growth factors in response to environmental stimuli such as hypoxia, Bussolino, F., Trends Biochem. Sci., 22, 251-256, (1997). These factors diffuse to nearby endothelial cells and stimulate receptors that lead to the production and secretion of proteases that degrade the surrounding extracellular matrix, Raza, S. L. et al, J. Investig. Dermatol. Symp. Proc., 5, 47-54, (2000); Stetler-Stevenson, W. G., Surg. Oncol. Clin. N. Am., 10, 383-392, (2001). These activated endothelial cells begin to migrate and proliferate into the surrounding tissue toward the source of these growth factors, Bussolino, F., Trends Biochem. Sci., 22, 251-256, (1997). Endothelial cells then stop proliferating and differentiate into tubular structures, which is the first step in the formation of stable, mature blood vessels, Glienke, J. et al, Eur. J. Biochem., 267, 2820-2830, (2000). Subsequently, periendothelial cells, such as pericytes and smooth muscle cells, are recruited to the newly formed vessel in a further step toward vessel maturation.

Angiogenesis is highly regulated by a delicate balance of naturally occurring pro- and anti-angiogenic factors, Folkman, J., J. Nat. Med., 1, 27-31, (1995). Vascular endothelial growth factor, fibroblast growth factor, and angiopoeitin represent a few of the many potential pro-angiogenic growth factors, Bussolino, F., Trends Biochem. Sci., 22, 251-256, (1997). These ligands bind to their respective receptor tyrosine kinases on the endothelial cell surface and transduce downstream signals that promote cell migration and proliferation, Mustonen, T. et al, J. Cell. Biol., 129, 895-898, (1995). Whereas a number of these regulatory factors have been identified, the molecular mechanisms of this process are still not fully understood.

There are many disease states driven by persistent unregulated angiogenesis. In such disease states, unregulated angiogenesis can either cause a particular disease directly or exacerbate an existing pathological condition. For example, ocular neovascularization has been implicated as the most common cause of blindness and underlies the pathology of approximately 20 eye diseases. In certain previously existing conditions such as arthritis, newly formed capillary blood vessels invade the joints and destroy cartilage. In diabetes, new capillaries formed in the retina invade the vitreous humor, causing bleeding and blindness.

Both the growth and metastasis of solid tumors are also angiogenesis-dependent, Folkman, J. Cancer Res., 46, 467-73 (1986); Folkman, J. Nat. Cancer Inst., 82, 4-6 (1989); Folkman et al., “Tumor Angiogenesis,” Chapter 10, 206-32, in The Molecular Basis of Cancer, Mendelsohn et al., eds., W. B. Saunders, (1995). It has been shown, for example, that tumors which enlarge to greater than 2 mm. in diameter must obtain their own blood supply and do so by inducing the growth of new capillary blood vessels. After these new blood vessels become embedded in the tumor, they provide nutrients and growth factors essential for tumor growth as well as a means for tumor cells to enter the circulation and metastasize to distant sites, such as liver, lung or bone, Weidner, New Eng. J. Med., 324, 1, 1-8 (1991). When used as drugs in tumor-bearing animals, natural inhibitors of angiogenesis can prevent the growth of small tumors, O'Reilly et al., Cell, 79, 315-28 (1994). Indeed, in some protocols, the application of such inhibitors leads to tumor regression and dormancy even after cessation of treatment, O'Reilly et al., Cell, 88, 277-85 (1997). Moreover, supplying inhibitors of angiogenesis to certain tumors can potentiate their response to other therapeutic regimens (e.g., chemotherapy) (see, e.g., Teischer et al., Int. J. Cancer, 57, 920-25 (1994)).

Although many disease states are driven by persistent unregulated angiogenesis, many disease states could be treated by increased angiogenesis. Tissue growth and repair are biologic events wherein cellular proliferation and angiogenesis occur. Thus an important aspect of wound repair is the revascularization of damaged tissue by angiogenesis.

Impaired tissue healing is a significant problem in health care. Chronic, non-healing wounds are a major cause of prolonged morbidity in the aged human population. This is especially the case in bedridden or diabetic patients who develop severe, non-healing skin ulcers. In many of these cases, the delay in healing is a result of inadequate blood supply either as a result of continuous pressure or of vascular blockage. Poor capillary circulation due to small artery atherosclerosis or venous stasis contribute to the failure to repair damaged tissue. Such tissues are often infected with microorganisms that proliferate unchallenged by the innate defense systems of the body which require well vascularized tissue to effectively eliminate pathogenic organisms. As a result, most therapeutic intervention centers on restoring blood flow to ischemic tissues thereby allowing nutrients and immunological factors access to the site of the wound.

Atherosclerotic lesions in large vessels can cause tissue ischemia that could be ameliorated by modulating blood vessel growth to supply the affected tissue. For example, atherosclerotic lesions in the coronary arteries cause angina and myocardial infarction that could be prevented if one could restore blood flow by stimulating the growth of collateral arteries. Similarly, atherosclerotic lesions in the large arteries that supply the legs cause ischemia in the skeletal muscle that limits mobility and in some cases necessitates amputation which could also be prevented by improving blood flow with angiogenic therapy.

Other diseases such as diabetes and hypertension are characterized by a decrease in the number and density of small blood vessels such as arterioles and capillaries. These small blood vessels are critical for the delivery of oxygen and nutrients and any decrease in the number and density of these vessels contributes to the adverse consequences of hypertension and diabetes including claudication, ischemic ulcers, accelerated hypertension, and renal failure. These common disorders and many other less common ailments such as Burgers disease would be ameliorated by increasing the number and density of small blood vessels using angiogenic therapy.

Thus, there is a continuing need to identify modulators of angiogenesis.

### SUMMARY OF THE INVENTION

The present invention is based upon the discovery of protein expression profiles using a rat cornea model at various stages of angiogenesis. As such, the present invention identifies and provides proteins that are effective in modulating angiogenesis. To this end, the present invention provides for methods, compositions, and kits comprising a safe and effective amount of an angiogenesis modulating protein (“AMP”) that may be used for the treatment of an angiogenesis mediated disorder.

One aspect of the invention provides for a method of screening an agent useful for treating an angiogenesis mediated disorder, suitable for high throughput screening, comprising the steps of: (a) exposing an AMP to the agent; and (b) measuring activity of AMP; wherein a modulation in AMP activity indicates the agent is useful for treating the angiogenesis mediated disorder.

Another aspect of the invention provides for a method of screening an agent useful for treating an angiogenesis mediated disorder comprising the steps of: (a) exposing an AMP to the agent; (b) measuring binding of the agent to the AMP; wherein binding of the agent to the AMP indicates the agent is useful for treating the angiogenesis mediated disorder.

Another aspect of the invention provides for a method of screening an agent useful for treating an angiogenesis mediated disorder comprising the steps of: (a) exposing an AMP encoding nucleotide sequence to the agent; (b) measuring the binding of the agent to the AMP encoding nucleotide sequence; wherein binding of the agent to the AMP encoding nucleotide sequence indicates the agent is useful for treating the angiogenesis mediated disorder.

Another aspect of the invention provides for a method of screening an agent useful for treating an angiogenesis mediated disorder, suitable for a cell-based assay, comprising the steps of: (a) exposing a cell to the agent; and (b) measuring expression or activity of AMP in the cell; wherein a modulation in the expression or the activity of AMP indicates the agent is useful for the treatment of the angiogenesis mediated disorder. In one embodiment, the expression is mRNA expression (i.e., transcription). In another embodiment, the expression is protein expression (i.e., translation).

Another aspect of the invention provides for a method of screening an agent useful for treating an angiogenesis mediated disorder comprising the steps of: (a) exposing a cell to the agent; and (b) measuring the association of the agent and AMP; wherein a modulation in the association of the agent and AMP indicates the agent is useful for the treatment of the angiogenesis mediated disorder.

One aspect of the invention provides for a pharmaceutical composition comprising: (a) a safe and effective amount of AMP or a nucleotide sequence encoding the same; and (b) a pharmaceutically-acceptable carrier.

Another aspect of the invention provides for a pharmaceutical composition comprising: (a) a safe and effective amount of an agent that modulates AMP expression or activity; and (b) a pharmaceutically-acceptable carrier.

One aspect of the invention provides for a method of treating an angiogenesis mediated disorder in a subject in need thereof by administering a safe and effective amount of AMP or a nucleotide sequence encoding the same.

Another aspect of the invention provides for a method of treating an angiogenesis mediated disorder in a subject in need thereof by administering a safe and effective amount of an agent that modulates AMP expression or activity.

Another aspect of the invention provides for a method of diagnosing or monitoring status of an angiogenesis mediated disorder in a subject comprising the steps of: (a) obtaining a cell sample from the subject; (b) measuring expression or activity of AMP in the cell; wherein a modulation in the expression or the activity of AMP indicates the diagnoses or status of the angiogenesis mediated disorder in the subject, respectively.

One aspect of the invention provides for an agent identified by any of the methods of the present invention.

### BRIEF DESCRIPTION OF THE FIGURES

FIGS. 1-5. Photomicrographs of ink-filled vessels in the rat corneas. Corneas are cauterized for 5 seconds with a silver nitrate applicator stick and harvested at days 0, 2, 4, 7, and 15 post-cautery (FIGS. 1-5, respectively). Animals are perfused with ink prior to harvest to visualize the vasculature. Only the normal limbal vasculature is seen in the Day 0, non-cauterized cornea (FIG. 1). A dense brushwork of new vessels sprouting from the surrounding limbal vessels can be seen in 48 hours (FIG. 2). Vessels continue to elongate toward the site of cautery on Day 4 (FIG. 3). Between Days 6 and 7, regression and remodeling occur as redundant vessels are “pruned” (FIG. 4) until only a few stable, mature vessels remain in the cornea as seen on Day 15 (FIG. 5).

FIGS. 6-7. Comparison of representative 2-DE (two-dimensional gel electrophoresis) profiles from control and angiogenic rat corneas (FIGS. 6 and 7, respectively). Proteins are solubilized from control and angiogenic (Day 3 post-cautery) rat corneas and separated in the first dimension by IEF (iso-electric focusing) using carrier ampholytes, pH 4-7. Separation in the second dimension is performed using 10% tricine gels followed by polychromatic silver staining. Differentially-expressed proteins are excised from gels and identified by mass spectrometry. All identified proteins are numbered and listed in Table II.

FIGS. 8-13. Representative 2-DE profiles of rat comeas from a time course study of angiogenesis. Proteins are solubilized from corneas harvested on Days 0, 2, 4, 6, 7, and 15 post-cautery (FIGS. 8-13, respectively). Extracted proteins are separated in the first dimension by EF (iso-electric focusing) using carrier ampholytes, pH 4-7. Separation in the second dimension is performed using 10% tricine gels followed by polychromatic silver staining. Differentially-expressed proteins are excised from gels and identified by mass spectrometry. All identified proteins are numbered and listed in Table III.

### SEQUENCE LISTING DESCRIPTION

Each of the nucleotide and protein sequences in the sequence listing, along with the corresponding Genbank or Derwent accession number(s) and animal species from which it is cloned, is shown in Table I.

TABLE I

| | | | Genbank | |
|---|---|---|---|---|
| | | | (GB) or | |
| | | | Derwent | |
| | | | (D) | Related |
| | SEQ ID | | Accession | Genbank (GB) |
| | NO: | | No. for | or Derwent |
| Sequence | nucleotide, | | nucleotide | (D) Accession |
| description | amino acid | Species | sequence | Nos. |
| CRP55 | 1, 2 | Rattus | X53363 | 117505 |
| | | Norvegicus | | 55854 |
| | | | | 1089798 |
| | | | | 488840 |
| CRP55 | 3, 4 | Rattus | D78308 | |
| | | Norvegicus | | |
| CRP55 | 5, 6 | Rattus | X79327 | |
| | | Norvegicus | | |
| Keratin | 7, 8 | Mus | U02880 | 7110667 |
| Complex I | | Musculus | | 414539 |
| | | | | 565659 |
| Keratin | 9, 10 | Mus | U08095 | |
| Complex I | | Musculus | | |
| Heat Shock 60 | 11, 12 | Rattus | X53585 | 111757 |
| Precursor | | Norvegicus | | 56380 |
| | | | | 56382 |
| Heat Shock 60 | 13, 14 | Rattus | X54793 | |
| Precursor | | Norvegicus | | |
| Aldehyde | 15, 16 | Rattus | J0367 | 118507 |
| Dehyrogenase | | Norvegicus | | 202832 |
| | | | | 202845 |
| Aldehyde | 17, 18 | Rattus | M23995 | |
| Dehyrogenase | | Norvegicus | | |
| Capping | 19, 20 | Mus | Z93101 | 6680842 |
| Protein | | Musculus | | 1903235 |
| | | | | 500748 |
| | | | | 500746 |
| Capping | 21, 22 | Mus | U10407 | |
| Protein | | Musculus | | |
| Capping | 23, 24 | Mus | U10406 | |
| Protein | | musculus | | |
| Alcohol | 25, 26 | Rattus | X98746 | 627982 |
| Dehydrogenase | | Norvegicus | | 4379399 |
| Heat Shock 70 | 27, 28 | Rattus | L16764 | 631840 |
| | | Norvegicus | | 294567 |
| | | | | 1483577 |
| Heat Shock 70 | 29, 30 | Rattus | Z75029 | |
| | | Norvegicus | | |
| Ribosomal | 31, 32 | Rattus | M57428 | M35864(GB) |
| Protein | | Norvegicus | | 2117822 |
| S6 Kinase | | | | 206839 |
| | | | | 206841 |
| Ribosomal | 33, 34 | Rattus | M58340 | M37777(GB) |
| Protein | | Norvegicus | | |
| S6 Kinase | | | | |
| Serine Protease | 35, 36 | Rattus | X16357 | 92743 |
| Inhibitor I | | Norvegicus | | 57230 |
| | | | | 207041 |
| Serine Protease | 37, 38 | Rattus | M15917 | J02692(GB) |
| Inhibitor I | | Norvegicus | | |
| Hemopexin | 39, 40 | Rattus | M62642 | J05306(GB) |
| Precursor | | Norvegicus | | 123036 |
| | | | | 554442 |
| Serum | 41, 42 | Rattus | V01222 | J00698(GB) |
| Albumin | | Norvegicus | | 113580 |
| Precursor | | | | 55627 |
| Myosin Heavy | 43, 44 | Rattus | X16262 | 92511 |
| Chain | | Norvegicus | | 56650 |
| Calgranulin A | 45, 46 | Rattus | L18891 | 1710812 |
| | | Norvegicus | | 349548 |
| Kallikrein- | 47, | Rattus | M19647 | J02837(GB) |
| binding | 48-80 | Norvegicus | | 92335 |
| Protein | | | | 204999 |
| Precursor | | | | 205010 |
| Kallikrein- | 81, 82 | Rattus | M30282 | |
| binding | | Norvegicus | | |
| Protein | | | | |
| Precursor | | | | |
| α-2-HS | 83, 84 | Rattus | M29758 | 231468 |
| Glycoprotein | | Norvegicus | | 951426 |
| Precursor | | | | 56139 |
| α-2-HS | 85, 86 | Rattus | X63446 | |
| Glycoprotein | | Norvegicus | | |
| Precursor | | | | |
| β-Actin | 87, 88 | Rattus | J00691 | V01217(GB) |
| | | Norvegicus | | 71620 |
| | | | | 55574 |
| Procollagen, | 89, 90 | Mus | Z18271 | 57956 |
| type V1, α1 | | Musculus | | 57955 |
| | | | | 50478 |
| Procollagen, | 91, 92 | Mus | X66405 | |
| type V1, α1 | | Musculus | | |
| Collagen α 3 | 93 | Mus | Z81279 | 4104232 |
| | | Musculus | | 57959 |
| | | | | 3236369 |
| Collagen α 3 | 94, 95 | Mus | AF064749 | |
| | | Musculus | | |
| HSP70 | 96, 97 | Rattus | X77207 | 2119721 |
| | | Norvegicus | | 1814000 |
| Reticulocalbin | 98, 99 | Mus | D13003 | 6677691 |
| | | Musculus | | 220581 |
| Macrophage | 100, 101 | Mus | X54511 | 729023 |
| Capping | | Musculus | | 53017 |
| Protein | | | | 18605628 |
| Macrophage | 102, 103 | Mus | BC023101 | |
| Capping | | Musculus | | |
| Protein | | | | |
| Phosphatidyl | 104, 150 | Rattus | X75253 | 8393910 |
| Ethanolamine | | Norvegicus | | 406291 |
| Binding Protein | | | | 510338 |
| Phosphatidyl | 106, 107 | Rattus | X71873 | |
| Ethanolamine | | Norvigcus | | |
| Binding Protein | | | | |
| Heat Shock 27 | 108, 109 | Rattus | S67755 | 478327 |
| | | Norvegicus | | 544757 |
| α-Crystalline A | 110, 111 | Rattus | U47921 | 7106271 |
| Chain | | Norvegicus | | 1245159 |
| | | | | 1245161 |
| α-Crystalline A | 112, 113 | Rattus | U47922 | |
| Chain | | Norvegicus | | |
| Gelsolin | 114, 115- | Sus Scrofa | M36927 | 121118 |
| Precursor | 133 | | | 1951 |
| | | | | 164471 |
| Gelsolin | 134, 135- | Sus Scrofa | X13871 | |
| Precursor | 153 | | | |
| Crystatin-β | 154, 155 | Rattus | X54737 | 6978715 |
| | | Norvegicus | | 55891 |
| Vitamin D | 156, 157 | Rattus | J05148 | 203941 |
| Binding Protein | | Norvegicus | | 203940 |
| Precursor | | | | 203926 |
| Vitamin D | 158,159 | Rattus | M12450 | |
| Binding Protein | | Norvegicus | | |
| Precursor | | | | |
| Tropomyosin 4 | 160, 161 | Rattus | J02780 | 6981672 |
| | | Norvegicus | | 207503 |
| Tropomyosin | 162, 163 | Rattus | L00372 | M12080(GB) |
| | | Norvegicus | | M14127(GB) |
| | | | | 136073 |
| | | | | 207485 |
| | | | | 207495 |
| Tropomyosin | 164, 165 | Rattus | L00373 | M12080(GB) |
| | | Norvegicus | | M14127(GB) |
| Tropomyosin | 166, 167 | Rattus | L00374 | M12080(GB) |
| | | Norvegicus | | M14127(GB) |
| Tropomyosin | 168, 169- | Rattus | L00375 | M12080(GB) |
| | 172 | Norvegicus | | M14127(GB) |
| Tropomyosin | 173, 174 | Rattus | L00376 | M12080(GB) |
| | | Norvegicus | | M14127(GB) |
| Tropomyosin | 175, 176 | Rattus | L00377 | M12080(GB) |
| | | Norvegicus | | M14127(GB) |
| Tropomyosin | 177, 178 | Rattus | L00378 | M12080(GB) |
| | | Norvegicus | | M14127(GB) |
| Tropomyosin | 179, 180 | Rattus | L00379 | M12080(GB) |
| | | Norvegicus | | M14127(GB) |
| Tropomyosin | 181, 182 | Rattus | L00380 | M12080(GB) |
| | | Norvegicus | | M14127(GB) |
| Tropomyosin | 183, 184- | Rattus | L00381 | M12080(GB) |
| | 185 | Norvegicus | | M14127(GB) |
| Tropomyosin | 186, 187 | Rattus | L00382 | M12080(GB) |
| | | Norvegicus | | M14127(GB) |
| Transthyretin | 188, 189 | Rattus | K03251 | 136467 |
| Precursor | | Norvegicus | | 205983 |
| (prealbumin) | | | | 57424 |
| | | | | 205981 |
| Transthyretin | 190, 191 | Rattus | X14876 | |
| Precursor | | Norvegicus | | |
| (prealbumin) | | | | |
| Transthyretin | 192, 193 | Rattus | K03252 | |
| Precursor | | Norvegicus | | |
| (prealbumin) | | | | |
| Vimentin | 194 | Rattus | X62951 | 401365 |
| | | Norvegicus | | 56859 |
| | | | | 57479 |
| | | | | 56908 |
| Vimentin | 195, 196 | Rattus | X62952 | |
| | | Norvegicus | | |
| Vimentin | 197 | Rattus | X62953 | |
| | | Norvegicus | | |
| Fatty Acid | 198, 199 | Rattus | S69874 | 1706754 |
| Binding Protein | | Norvegicus | | 546419 |
| | | | | 1836057 |
| | | | | 533123 |
| Fatty Acid | 200, 201 | Rattus | U13253 | |
| Binding Protein | | Norvegicus | | |
| Fatty Acid | 202, 203 | Rattus | S83247 | |
| Binding Protein | | Norvegicus | | |
| Eukaryotic | 204, 205 | Rattus | AF304351 | 9055210 |
| Translation | | Norvegicus | | 10442751 |
| Elongation | | | | 15341783 |
| Factor | | | | 18605703 |
| | | | | 14708537 |
| Eukaryotic | 206, 207 | Rattus | BC023139 | |
| Translation | | Norvegicus | | |
| Elongation | | | | |
| Factor | | | | |
| Eukaryotic | 208, 209- | Rattus | BC003969 | |
| Translation | 211 | Norvegicus | | |
| Elongation | | | | |
| Factor | | | | |
| Eukaryotic | 212, 213 | Rattus | BC013059 | |
| Translation | | Norvegicus | | |
| Elongation | | | | |
| Factor | | | | |
| t-Kininogen | 214, 215 | Rattus | M11883 | 69856615 |
| | | Norvegicus | | 205084 |
| | | | | 205305 |
| t-Kininogen | 216, 217 | Rattus | X02299 | |
| | | Norvegicus | | |
| t-Kininogen | 218, 219- | Rattus | K02814 | |
| | 236 | Norvegicus | | |
| Apolipoprotein | 237, 238 | Rattus | J02588 | 114008 |
| A-IV Precursor | | Norvegicus | | 2029327 |
| | | | | 202942 |
| | | | | 202949 |
| Apolipoprotein | 239, 240 | Rattus | M13508 | |
| A-IV Precursor | | Norvegicus | | |
| Apolipoprotein | 241, 242 | Rattus | M00002 | |
| A-IV Precursor | | Norvegicus | | |
| Preprohapto- | 243, 244- | Rattus | K01933 | 204657 |
| globin | 258 | Norvegicus | | 204656 |
| Galectin 7 | 259, 260 | Homo | L07769 | 4504985 |
| | | Sapiens | | 182131 |
| Lipocortin-III | 261, 262 | Rattus | M20559 | J03898(GB) |
| | | Norvegicus | | 6978503 |
| | | | | 205136 |
| Pyruvate | 263, 264 | Rattus | M24359 | 1346398 |
| Kinase | | Norvegicus | | 206203 |
| | | | | 206206 |
| Pyruvate | 265 | Rattus | M14377 | |
| Kinase | | Norvegicus | | |
| α-1-macro- | 266, 267 | Rattus | M77183 | 202857 |
| globulin | | Norvegicus | | 202856 |
| | | | | 205383 |
| α-1-macro- | 268, 269 | Rattus | M84000 | J05359(GB) |
| globulin | | Norvegicus | | |
| 40S Ribosomal | 270, 271 | Rattus | D25224 | 631907 |
| Protein P40 | | Norvegicus | | 466438 |
| Serotransferrin | 272, 273 | Rattus | X77158 | 6175089 |
| Precursor | | Norvegicus | | 510195 |
| | | | | 1854475 |
| Serotransferrin | 274, 275 | Rattus | D38380 | |
| Precursor | | Norvegicus | | |
| Apolipoprotein | 276, 277 | Rattus | J02597 | 2145143 |
| A-1 | | Norvegicus | | 202935 |
| | | | | 2145146 |
| | | | | 2145144 |
| | | | | 2145142 |
| Apolipoprotein | 278, 279 | Rattus | U79576 | |
| A-1 | | Norvegicus | | |
| Apolipoprotein | 280, 281 | Rattus | U79577 | |
| A-1 | | Norvegicus | | |
| Apolipoprotein | 282, 283 | Rattus | U79578 | |
| A-1 | | Norvegicus | | |
| Apolipoprotein | 284, 285 | Rattus | J02597 | 113997 |
| A-1 Precursor | | Norvegicus | | 202944 |
| | | | | 55746 |
| | | | | 202935 |
| Apolipoprotein | 286, 287 | Rattus | M00001 | |
| A-1 Precursor | | Norvegicus | | |
| Apolipoprotein | 288, 289 | Rattus | X00558 | |
| A-1 Precursor | | Norvegicus | | |
| Thioredoxin | 290, 291 | Rattus | X14878 | 135776 |
| | | Norvegicus | | 57385 |
| Malate | 292, 293 | Rattus | AF093773 | 3747085 |
| Dehydrogenase | | Norvegicus | | 3747084 |
| α-2U-globulin | 294, 295 | Rattus | X14552 | 111366 |
| Precursor | | Norvegicus | | 55569 |
| | | | | 204262 |
| α-2U-globulin | 296, 297 | Rattus | J00738 | |
| Precursor | | Norvegicus | | |
| Lipocortin-1 | 298 | Rattus | S57478 | J05339(GB) |
| | | Norvegicus | | 235879 |
| | | | | 235878 |
| | | | | 56565 |
| Lipocortin-1 | 299, 300 | Rattus | Y00446 | |
| | | Norvegicus | | |
| Phospho- | 301, 302 | Mus | M15668 | 6679291 |
| glycerate | | Musculus | | 202422 |
| Kinase | | | | 341094 |
| Phospho- | 303, 304 | Mus | M23962 | |
| glycerate | | Musculus | | |
| Kinase | | | | |
| RHO GDP | 305, 306 | Mus | AB055070 | 2494703 |
| | | Musculus | | 193461 |
| | | | | 12597248 |
| RHO GOP | 307, 308 | Mus | L07918 | |
| | | Musculus | | |

### DETAILED DESCRIPTION OF THE INVENTION

### I. AMPs

The present invention is based on the discovery of differentially expressed proteins in protein expression profiles from various stages of angiogenesis. As used herein, “angiogenesis” means the formation of new blood vessels from pre-existing vasculature. The proteins, peptides, polypeptides, or nucleotide sequences encoding the same, of the present invention, are referred to herein collectively, unless indicated otherwise, as Angiogenesis Modulating Proteins (“AMPs”), and may be used as modulators of angiogenesis. As used herein, “modulate angiogenesis,” means to modify angiogenesis. The modulation of angiogenesis, as used herein, encompasses both the stimulation and inhibition of angiogenesis. As used herein, “stimulation of angiogenesis,” means to beneficially enhance or augment a naturally occurring angiogenic process or, alternatively, induce or initiate an angiogenic process. As used herein, “inhibition of angiogenesis,” means to beneficially reduce or diminish either a naturally occurring angiogenic process or disease/condition related angiogenic process or, alternatively, reduce or diminish the initiation of a naturally or disease/condition related angiogenic process.

The AMPs of the present invention are included in Table I.

Variants of AMP, are also encompassed by the present invention. As used herein, “variants,” means those proteins, peptides, or polypeptides, or nucleotide sequences encoding the same, that are substantially similar to those described by Table I and which may be used as to modulate angiogenesis. An AMP of Table I may be altered in various ways to yield a variant encompassed by the present invention including amino acid substitutions, deletions, truncations, insertions, and modifications. Methods for such manipulations are generally known in the art. For example, variants can be prepared by mutations in the nucleotide sequence. Methods for mutagenesis and nucleotide sequence alterations are well known in the art. See, for example, Kunkel, Proc. Natl. Acad. Sci. USA, 82, 488-492 (1985); Kunkel et al., Methods in Enzymol., 154, 367-382, (1987); U.S. Pat. No. 4,873,192; Walker and Gaastra, eds., Techniques in Molecular Biology, MacMillan Publishing Company, New York, (1983), and the references cited therein. In one embodiment of the variant, the substitution(s) of the AMP of Table I is conservative in that it minimally disrupts the biochemical properties of the variant. Thus, where mutations are introduced to substitute amino acid residues, positively charged residues (H, K, and R) preferably are substituted with positively-charged residues; negatively-charged residues (D and E) preferably are substituted with negatively-charged residues; and neutral non-polar residues (A, F, I, L, M, P, V, and W) preferably are substituted with neutral non-polar residues. In another embodiment of the variant, the overall charge, structure or hydrophobic/hydrophilic properties of the AMP can be altered without substantially adversely affecting the angiogenesis modulating capacity. In still another embodiment, the variant is an active fragment of an AMP of Table I. In yet another embodiment of the variant, an AMP of Table I is modified by acetylation, carboxylation, phosphorylation, glycosylation, ubiquitination, and labeling, whether accomplished by in vivo or in vitro enzymatic treatment of the AMP or by the synthesis of the AMP using modified amino acids. Non-limiting examples of modifications to amino acids include phosphorylation of tyrosine, serine, and threonine residues; methylation of lysine residues; acetylation of lysine residues; hydroxylation of proline and lysine residues; carboxylation of glutamic acid residues; glycosylation of serine, threonine, or asparagine residues; and ubiquitination of lysine residues. The variant can also include other domains, such as epitope tags and His tags (e.g., the protein can be a fusion protein).

In yet another embodiment, peptide mimics of an AMP of Table I are encompassed within the meaning of variant. As used herein, “mimic,” means an amino acid or an amino acid analog that has the same or similar functional characteristics of an amino acid. Thus, for example, an arginine analog can be a mimic of arginine if the analog contains a side chain having a positive charge at physiologic pH, as is characteristic of the guanidinium side chain reactive group of arginine. Examples of organic molecules that can be suitable mimics are listed at Table 1 of U.S. Pat. No. 5,807,819. Generally, a peptide variant, or nucleic acid sequence encoding the same, of preferably 99% sequence identity to its respective native amino acid sequence. Fusion proteins, or N-terminal, C-terminal or internal extensions, deletions, or insertions into the peptide sequence shall not be construed as affecting homology.

“Sequence Identity” or “Homology” at the amino acid or nucleotide sequence level is determined by BLAST (Basic Local Alignment Search Tool) analysis using the algorithm employed by the programs blastp, blastn, blastx, tblastn and tblastx, Altschul et al., Nucleic Acids Res. 25, 3389-3402 (1997) and Karlin et al. Proc. Natl. Acad. Sci., USA, 87, 2264-2268 (1990) which are tailored for sequence similarity searching. The approach used by the BLAST program is to first consider similar segments, with gaps (non-contiguous) and without gaps (contiguous), between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. Nature Genetics, 6, 119-129 (1994). The search parameters for histograms, descriptions, alignments, (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix, Henikoff et al. Proc. Natl. Acad. Sci. USA 89, 10915-10919 (1992), recommended for query sequences over 85 nucleotides or amino acids in length.

For blastn, the scoring matrix is set by the ratios of M (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

For the sake of brevity, the use of the term “AMP” hereinafter includes any “variant” thereof.

### II. Methods of Use

As discussed, the AMPs of the present invention are modulators of angiogenesis. As such, the AMPs (or agents that modulate AMP) of the present invention may be used in the prevention and treatment of angiogenesis mediated disorders. As used herein, “angiogenesis mediated disorders” include: (1) those disorders, diseases and/or unwanted conditions which are characterized by unwanted or elevated angiogenesis referred to herein collectively as “angiogenesis elevated disorders;” or (2) those disorders, diseases and/or unwanted conditions which are characterized by wanted or reduced angiogenesis referred to herein collectively as “angiogenesis reduced disorders.”

As used herein, “treatment,” means, at a minimum, administration of an AMP (or agent) of the present invention that mitigates an angiogenesis mediated disorder in a mammalian subject, preferably in humans. Thus, the term “treatment” includes preventing an angiogenesis mediated disorder in a mammal, particularly when the mammal is predisposed to acquiring the disorder, but has not yet been diagnosed with the disorder; inhibiting the disorder; and/or alleviating or reversing the disorder. It is understood that the term “prevent” does not require that the disease state be completely thwarted. (See Webster's Ninth Collegiate Dictionary.) Rather, as used herein, the term preventing refers to the ability of the skilled artisan to identify a population that is susceptible to angiogenesis mediated disorders, such that administration of the AMPs of the present invention may occur prior to the onset of the disease. The term does not imply that the disease state be completely avoided.

In accordance with the inventive method, AMP is provided to cells, preferably endothelial cells, associated with the tissue of interest. Such cells can be cells comprising the tissue of interest, exogenous cells introduced into the tissue, or neighboring cells not within the tissue. Thus, for example, the cells can be cells of the tissue, and AMP is provided to them in situ such that the AMP contacts the cells. Alternatively, the cells can be cells introduced into the tissue, in which case AMP can be transferred to the cells before they are introduced into the tissue (e.g., in vitro), as well as being transferred in situ after introduction into the tissue. The tissue with which the endothelial cells are associated is any tissue in which it is desired to modulate angiogenesis.

In one aspect of the invention, the method involves providing AMP by supplying an AMP to the cells (e.g., within a suitable composition). Any suitable method can be employed to obtain an AMP. Many suitable AMPs can be purified from tissues which naturally produce AMP or from media conditioned by a variety of AMP-producing cells (e.g., endothelial cells, smooth muscle cells, fibroblasts or parenchymal cells). Alternatively, AMP may be synthesized using standard direct peptide synthesizing techniques (e.g., as summarized in Bodanszky, Principles of Peptide Synthesis, Springer-Verlag, Heidelberg: (1984)), such as via solid-phase synthesis (see, e.g., Merrifield, J. Am. Chem. Soc., 85, 2149-54 (1963); Barany et al., Int. J. Peptide Protein Res., 30, 705-739 (1987); and U.S. Pat. No. 5,424,398). Of course, as genes for AMP are known (see Table I for Genbank or Derwent Accession Nos.); or can be deduced from the polypeptide sequences discussed herein. An AMP can be produced by standard recombinant methods.

In other protocols, AMP can be provided to the tissue of interest by transferring an expression cassette, including a nucleic acid encoding AMP, to cells associated with the tissue of interest. The cells produce and secrete the AMP such that it is suitably provided to endothelial cells within the tissue to modulate angiogenesis within the tissue of interest. As discussed, coding sequences for AMP are known and others can be deduced. Thus, AMP expression cassettes typically employ coding sequences homologous to these known sequences, e.g., they will hybridize to at least a fragment of the known sequences under at least mild stringency conditions, more preferably under moderate stringency conditions, most preferably under high stringency conditions (employing the definitions of mild, moderate, and high stringency as set forth in Sambrook et al., Molecular Cloning: A Laboratory Manual, 2d edition, Cold Spring Harbor Press (1989)).

In addition to the AMP coding sequence, an expression cassette includes a promoter, and, in the context of the present invention, the promoter must be able to drive the expression of the AMP gene within the cells. Many viral promoters are appropriate for use in such an expression cassette (e.g., retroviral ITRs, LTRs, immediate early viral promoters (IEp) (such as herpesvirus IEp (e.g., ICP4-IEp and ICP0-IEp) and cytomegalovirus (CMV) IEp), and other viral promoters (e.g., late viral promoters, latency-active promoters (LAPs), Rous Sarcoma Virus (RSV) promoters, and Murine Leukemia Virus (MLV) promoters)). Other suitable promoters are eukaryotic promoters, such as enhancers (e.g., the rabbit beta-globin regulatory elements), constitutively active promoters (e.g., the beta-actin promoter, etc.), signal specific promoters (e.g., inducible and/or repressible promoters, such as a promoter responsive to TNF or RU486, the metallothionine promoter, etc.), and tumor-specific promoters.

Within the expression cassette, the AMP gene and the promoter are operably linked such that the promoter is able to drive the expression of the AMP gene. As long as this operable linkage is maintained, the expression cassette can include more than one gene, such as multiple genes separated by ribosome entry sites. Furthermore, the expression cassette can optionally include other elements, such as polyadenylation sequences, transcriptional regulatory elements (e.g., enhancers, silencers, etc.), or other sequences.

The expression cassette must be introduced into the cells in a manner suitable for them to express the AMP gene contained therein. Any suitable vector can be so employed, many of which are known in the art. Examples of such vectors include naked DNA vectors (such as oligonucleotides or plasmids), viral vectors such as adeno-associated viral vectors, Bems et al., Ann. N.Y Acad. Sci., 772, 95-104 (1995), adenoviral vectors, Bain et al., Gene Therapy, 1, S68 (1994), herpesvirus vectors, Fink et al., Ann. Rev. Neurosci., 19, 265-87 (1996), packaged amplicons, Federoff et al., Proc. Nat. Acad. Sci. USA, 89, 1636-40 (1992), pappiloma virus vectors, picornavirus vectors, polyoma virus vectors, retroviral vectors, SV40 viral vectors, vaccinia virus vectors, and other vectors. A non-limiting example of a suitable vector is disclosed in U.S. patent application Ser. No. 2001-0041679 A1.

In addition to the expression cassette of interest, the vector can also include other genetic elements, such as, for example, genes encoding a selectable marker (e.g., beta-gal or a marker conferring resistance to a toxin), a pharmacologically active protein, a transcription factor, or other biologically active substance.

Once a given type of vector is selected, its genome must be manipulated for use as a background vector, after which it must be engineered to incorporate exogenous polynucleotides. Methods for manipulating the genomes of vectors are well known in the art (see, e.g., Sambrook et al., supra) and include direct cloning, site specific recombination using recombinases, homologous recombination, and other suitable methods of constructing a recombinant vector. In this manner, the expression cassette can be inserted into any desirable position of the vector.

The vector harboring the AMP expression cassette is introduced into the cells by any means appropriate for the vector employed. Many such methods are well-known in the art (Sambrook et al., supra; see also Watson et al., Recombinant DNA, Chapter 12, 2d edition, Scientific American Books (1992)). Thus, plasmids are transferred by methods such as calcium phosphate precipitation, electroporation, liposome-mediated transfection, gene gun, microinjection, viral capsid-mediated transfer, polybrene-mediated transfer, protoplast fusion, etc. Viral vectors are best transferred into the cells by infecting them; however, the mode of infection can vary depending on the virus.

Cells into which the AMP gene has been transferred can be used in the inventive method as transient transformants. Alternatively, where the cells are cells in vitro, they can be subjected to several rounds of clonal selection (if the vector also contains a gene encoding a selectable marker, such as a gene conferring resistance to a toxin) to select for stable transformants.

Within the cells, the AMP gene is expressed such that the cells express and secrete AMP. Successful expression of the gene can be assessed via standard molecular biological techniques (e.g., Northern hybridization, Western blotting, immunoprecipitation, enzyme immunoassay, etc.). Reagents for detecting the expression of AMP genes and the secretion of AMP from transfected cells are known in the art (see, e.g., published international patent applications WO 95/33480 and WO 93/24529); Steele et al., supra).

Depending on the location of the tissue of interest, AMP can be supplied in any manner suitable to provide it to endothelial cells within the tissue of interest. Thus, for example, a composition containing a source of AMP (i.e., an AMP polypeptide or an AMP expression cassette, as described herein) can be introduced into the systemic circulation, which will distribute the source of AMP to the tissue of interest. Alternatively, a composition containing a source of AMP can be applied topically to the tissue of interest (e.g., injected as a bolus within a tumor or intercutaneous or subcutaneous site, applied to all or a portion of the surface of the skin, dropped onto the surface of the eye, etc.).

A. Treatment of Angiogenesis Elevated Disorder.

The AMPs (or agents) of the present invention may be used in a method for the treatment of an angiogenesis mediated disorder. In one aspect in the method for the treatment of an angiogenesis mediated disorder, an AMP may be used in a method for the treatment of an “angiogenesis elevated disorder.” As used herein, an “angiogenesis elevated disorder” is one that involves unwanted or elevated angiogenesis in the biological manifestation of the disease, disorder, and/or condition; in the biological cascade leading to the disorder; or as a symptom of the disorder. This “involvement” of angiogenesis in an angiogenesis elevated disorder includes, but is not limited to, the following: (1) The unwanted or elevated angiogenesis as a “cause” of the disorder or biological manifestation, whether the level of angiogenesis is elevated genetically, by infection, by autoimmunity, trauma, biomechanical causes, lifestyle, or by some other causes. (2) The angiogenesis as part of the observable manifestation of the disease or disorder. That is, the disease or disorder is measurable in terms of the increased angiogenesis. From a clinical standpoint, unwanted or elevated angiogenesis indicates the disease, however, angiogenesis need not be the “hallmark” of the disease or disorder. (3) The unwanted or elevated angiogenesis is part of the biochemical or cellular cascade that results in the disease or disorder. In this respect, inhibition of angiogenesis interrupts the cascade, and thus controls the disease. Non-limiting examples of angiogenesis reduced disorders that may be treated by the present invention are herein described below.

The AMPs of present invention may be used to treat diseases associated with retinal/choroidal neovascularization that include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy, whether or not associated with diabetes.

AMPs of the present invention can treat diseases associated with chronic inflammation. Diseases with symptoms of chronic inflammation include inflammatory bowel diseases such as Crohn's disease and ulcerative colitis, psoriasis, sarcoidosis and rheumatoid arthritis. Angiogenesis is a key element that these chronic inflammatory diseases have in common. The chronic inflammation depends on continuous formation of capillary sprouts to maintain an influx of inflammatory cells. The influx and presence of the inflammatory cells produce granulomas and thus, maintain the chronic inflammatory state. Inhibition of angiogenesis by the compositions and methods of the present invention would prevent the formation of the granulomas and alleviate the disease.

AMPs may used to treat patients with inflammatory bowel diseases such as Crohn's disease and ulcerative colitis. Both Crohn's disease and ulcerative colitis are characterized by chronic inflammation and angiogenesis at various sites in the gastrointestinal tract. Crohn's disease is characterized by chronic granulomatous inflammation throughout the gastrointestinal tract consisting of new capillary sprouts surrounded by a cylinder of inflammatory cells. Prevention of angiogenesis by the AMPs of the present invention inhibits the formation of the sprouts and prevents the formation of granulomas. Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea.

The inflammatory bowel diseases also show extraintestinal manifestations such as skin lesions. Such lesions are characterized by inflammation and angiogenesis and can occur at many sites other than the gastrointestinal tract. The AMPs of the present invention may be capable of treating these lesions by preventing the angiogenesis, thus reducing the influx of inflammatory cells and the lesion formation.

Sarcoidosis is another chronic inflammatory disease that is characterized as a multisystem granulomatous disorder. The granulomas of this disease may form anywhere in the body and thus the symptoms depend on the site of the granulomas and whether the disease active. The granulomas are created by the angiogenic capillary sprouts providing a constant supply of inflammatory cells.

AMPs of the present invention can also treat the chronic inflammatory conditions associated with psoriasis. Psoriasis, a skin disease, is another chronic and recurrent disease that is characterized by papules and plaques of various sizes. Prevention of the formation of the new blood vessels necessary to maintain the characteristic lesions leads to relief from the symptoms.

Another disease that may be treated according to the present invention, is rheumatoid arthritis. Rheumatoid arthritis is a chronic inflammatory disease characterized by nonspecific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Other diseases that can be treated according to the present invention are hemangiomas, Osler-Weber-Rendu disease, or hereditary hemorrhagic telangiectasia, solid or blood borne tumors and acquired immune deficiency syndrome.

B. Treatment of an Angiogenesis Reduced Disorder.

The AMPs (or agents) of the present invention may be used in a method for the treatment of an angiogenesis mediated disorder. In one aspect in the method for the treatment of an angiogenesis mediated disorder, an AMP may be used in a method for the treatment of an “angiogenesis reduced disorder.” As used herein, an “angiogenesis reduced disorder” is one that involves wanted or stimulated angiogenesis to treat a disease, disorder, and/or condition. The disorder is one characterized by tissue that is suffering from or be at risk of suffering from ischemic damage, infection, and/or poor healing, which results when the tissue is deprived of an adequate supply of oxygenated blood due to inadequate circulation. As used herein, “tissue” is used in the broadest sense, to include, but not limited to, the following: cardiac tissue, such as myocardium and cardiac ventricles; erectile tissue; skeletal muscle; neurological tissue, such as from the cerebellum; internal organs, such as the brain, heart, pancreas, liver, spleen, and lung; or generalized area of the body such as entire limbs, a foot, or distal appendages such as fingers or toes. This inadequate blood supply to tissue includes the following: (1) The inadequate blood supply as a “cause” of the disorder or the biological manifestation thereof, whether the level of blood supply is reduced genetically, by infection, by autoimmunity, trauma, surgery, biomechanical causes, lifestyle, or by some other causes. (2) The inadequate blood supply as part of the observable manifestation of the disorder. That is, the disorder is measurable in terms of the inadequate blood supply. From a clinical standpoint, inadequate blood supply indicates the disease, however, inadequate blood supply need not be the “hallmark” of the disorder. (3) The inadequate blood supply is part of the biochemical or cellular cascade that results in the disorder. In this respect, stimulation of angiogenesis interrupts the cascade, and thus controls the disorder. Non-limiting examples of angiogenesis reduced disorders that may be treated by the present invention are herein described below.

1. Method of Vascularizing Ischermic Tissue

In one aspect in the method for the treatment of an angiogenesis reduced disorders, an AMP (or agent) may be used in a method of vascularizing ischemic tissue. As used herein, “ischemic tissue,” means tissue that is deprived of adequate blood flow. Examples of ischemic tissue include, but are not limited to, tissue that lack adequate blood supply resulting from mycocardial and cerebral infarctions, mesenteric or limb ischemia, or the result of a vascular occlusion or stenosis. In one example, the interruption of the supply of oxygenated blood may be caused by a vascular occlusion. Such vascular occlusion can be caused by arteriosclerosis, trauma, surgical procedures, disease, and/or otheretiologies. There are many ways to determine if a tissue is at risk of suffering ischemic damage from undesirable vascular occlusion. Such methods are well known to physicians who treat such conditions. For example, in myocardial disease these methods include a variety of imaging techniques (e.g., radiotracer methodologies, x-ray, and MRI) and physiological tests. Therefore, induction of angiogenesis in tissue affected by or at risk of being affected by a vascular occlusion is an effective means of preventing and/or attenuating ischemia in such tissue. Thus, the treatment of skeletal muscle and myocardial ischemia, stroke, coronary artery disease, peripheral vascular disease, coronary artery disease are fully contemplated.

Any person skilled in the art of using standard techniques can measure the vascularization of tissue. Non-limiting examples of measuring vascularization in a subject include: SPECT (single photon emission computed tomography); PET (positron emission tomography); MRI (magnetic resonance imaging); and combination thereof, by measuring blood flow to tissue before and after treatment. Angiography can be used as an assessment of macroscopic vascularity. Histologic evaluation can be used to quantify vascularity at the small vessel level. These and other techniques are discussed in Simons, et al., “Clinical trials in coronary angiogenesis,” Circulation, 102, 73-86 (2000).

2. Method of Repairing Tissue

In one aspect in the method for the treatment of an angiogenesis reduced disorder, an AMP (or agent) may be used in a method of repairing tissue. As used herein, “repairing tissue” means promoting tissue repair, regeneration, growth, and/or maintenance including, but not limited to, wound repair or tissue engineering. One skilled in the art readily appreciates that new blood vessel formation is required for tissue repair. In turn, tissue may be damaged by, including, but not limited to, traumatic injuries or conditions including arthritis, osteoporosis and other skeletal disorders, and burns. Tissue may also be damaged by results from injuries due to surgical procedures, irradiation, laceration, toxic chemicals, viral infection bacterial infection or burns. Tissue in need of repair also includes non-healing wounds. Non-limiting examples of non-healing wounds include: non-healing skin ulcers resulting from diabetic pathology; or fractures that do not heal readily.

AMPs may also be used in a method to aid in tissue repair in the context of guided tissue regeneration (GTR) procedures. Such procedures are currently used by those skilled in the medical arts to accelerate wound healing following invasive surgical procedures.

AMPs may be used in a method of promoting tissue repair characterized by enhanced tissue growth during the process of tissue engineering. As used herein, “tissue engineering” is defined as the creation, design, and fabrication of biological prosthetic devices, in combination with synthetic or natural materials, for the augmentation or replacement of body tissues and organs. Thus, the present method can be used to augment the design and growth of human tissues outside the body for later implantation in the repair or replacement of diseased tissues. For example, AMPs may be useful in promoting the growth of skin graft replacements that are used as a therapy in the treatment of burns.

In another aspect of tissue engineering, AMPs of the present invention may be included in cell-containing or cell-free devices that induce the regeneration of functional human tissues when implanted at a site that requires regeneration. As previously discussed, biomaterial-guided tissue regeneration can be used to promote bone regrowth in, for example, periodontal disease. Thus, an AMP may be used to promote the growth of reconstituted tissues assembled into three-dimensional configurations at the site of a wound or other tissue in need of such repair.

In another aspect of tissue engineering, AMPs can be included in external or internal devices containing human tissues designed to replace the function of diseased internal tissues. This approach involves isolating cells from the body, placing them on or within structural matrices, and implanting the new system inside the body or using the system outside the body. The method of the invention can be included in such matrices to promote the growth of tissues contained in the matrices. For example, an AMP can be included in a cell-lined vascular graft to promote the growth of the cells contained in the graft. It is envisioned that the method of the invention can be used to augment tissue repair, regeneration and engineering in products such as cartilage and bone, central nervous system tissues, muscle, liver, and pancreatic islet (insulin-producing) cells.

### III. Methods of Screening an Agent Useful for Treating an Angiogenesis Mediated Disorder

The present invention is also based upon the surprising discovery of differential protein expression at various stages of angiogenesis using a rat cornea model of angiogenesis. In view of these surprising discoveries, AMPs may be used for screening agents useful in the treatment of angiogenesis mediated disorders in any of a variety of well-known drug screening techniques.

In one embodiment of the invention, AMPs can be used for screening libraries of agents in any of a variety of drug screening techniques. The AMP employed in such screening may be free in solution, affixed to a solid support, borne on a cell surface, or located intracellularly. The modulation of AMP activity or expression by the agent being tested may be measured.

Another technique for agent screening provides for high throughput screening of agents having suitable binding affinity to the protein or nucleotide of interest. (See, e.g., Geysen, et al. (1984) PCT application W084/03564.) In this method, large numbers of different test agents are synthesized on a solid substrate, such as plastic pins or some other surface. The test agents are reacted with AMP and washed. Bound AMP is then detected by methods well known in the art. Purified AMP can also be coated directly onto plates for use in the aforementioned drug screening techniques. Alternatively, non-neutralizing antibodies can be used to capture the AMP and immobilize it on a solid support.

In yet another embodiment, one may use competitive drug screening assays in which neutralizing antibodies capable of binding AMP specifically compete with a test agent for binding AMP. In this manner, antibodies can be used to detect the presence of any peptide which shares one or more antigenic determinants with AMP.

A. Sources of AMP

Isolated AMP can be obtained by methods well known in the art. For example, AMP may be synthesized using standard direct peptide synthesizing techniques (e.g., as summarized in Bodanszky, Principles of Peptide Synthesis, Springer-Verlag, Heidelberg: (1984)), such as via solid-phase synthesis (see, e.g., Merrifield, J. Am. Chem. Soc., 85, 2149-54 (1963) Roberge, J. Y. et al., Science 269: 202-204 (1995); Barany et al., Int. J. Peptide Protein Res., 30, 705-739 (1987); and U.S. Pat. No. 5,424,398). Of course, as genes for AMP are known, disclosed herein, or can be deduced from the polypeptide sequences discussed herein. AMP can be produced by standard recombinant methods. The proteins may be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography, chromatofocussing, selective precipitation with such substances as ammonium sulfate; and others (see, e.g., Scopes, Protein Purification: Principles and Practice (1982); U.S. Pat. No. 4,673,641; and Sambrook et all, supra). For example, the target protein can be purified using a standard anti-target antibody column. Ultrafiltration and diafiltration techniques, in conjunction with protein concentration, are also useful.

For a cell based assay in accordance with the present invention, cells comprising AMP are well known in the art or can be modified to comprise AMP by methods well known in the art. Suitable cells that naturally comprise AMP include, but are not limited to, liver, lung, skeletal muscle, and brain. Cell lines that comprise enhanced levels AMP may be either purchased commercially or constructed. Well-known methods of providing cells with AMP include incorporating an expression cassette, including a nucleic acid encoding AMP to cells of interest. Standard transfection or transformation methods can be used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 82 (Deutscher ed., 1990)).

B. Measuring Activity of AMP.

The activity of AMP can be measured by methods well known in the art. For example, if the AMP is a phophatase, phosphatase activity is measured. In one format, the phosphatase activity is measured using a fluorescent assay that generates a fluorescent signal when the substrate is acted upon by the enzyme. Other small molecule phosphatase substrates such as PNPP (para nitro phenyl phosphate) could also be used. These assay formats may be scaled-up for utilization in a high throughput screening assays using FRET (fluorescence resonance energy transfer) FP (Fluorescence polarization) or Malachite green assay. Another means of assaying for AMP phosphatase activity is to measure the loss of phosphorylation of its known target.

C. Measuring Expression of AMP.

The expression of AMP can be measured by methods well known in the art. In general, host cells that contain the nucleic acid sequence encoding AMP and that express AMP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of AMP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on AMP is preferred, but a competitive binding assay may be employed. These and other assays are well known in the art. (See, e.g., Hampton, R. et al. (1990) Serological Methods, a Laboratory Manual, APS Press, St Paul, Minn., Section IV; Coligan, J. E. et al. (1997 and periodic supplements) Current Protocols in Immunology, Greene Pub. Associates and Wiley-Interscience, New York, N.Y.; and Maddox, D. E. et al. (1983) J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding AMP include oligolabeling, nick translation, end-labeling, or PCR amplification using a labeled nucleotide. Alternatively, the sequences encoding AMP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

D. Agent

As used herein, the term “agent,” is used in the broadest sense, to include, without limitation: peptides, peptidomimetics, polypeptides, proteins, chemical compounds, nucleotides, antibodies, small molecules, vitamin derivatives, or carbohydrates. In one embodiment, the agent is an agonist. In another embodiment, the agent is an antagonist.

For example, an agent that may modulate AMP gene expression is a polynucleotide. The polynucleotide may be an antisense, a triplex agent, or a ribozyme. For example, an antisense may be directed to the structural gene region or to the promoter region of an AMP gene.

In another example, an agent that may modulate AMP translation is an antisense nucleic acid or ribozyme that could be used to bind to the AMP mRNA or to cleave it. Antisense RNA or DNA molecules bind specifically with a targeted gene's mRNA message, interrupting the expression of that gene's protein product.

In one format, in the screening for an agent that modulates the expression of AMP, the assay format is such that the cell lines that contain reporter gene fusions between the open reading frame defined by nucleotides encoding AMP and/or the 5′ and/or 3′ regulatory elements and any assayable fusion partner may be prepared. Numerous assayable fusion partners are known and readily available including the firefly luciferase gene and the gene encoding chloramphenicol acetyltransferase, Alam et al. Anal. Biochem., 188, 245-254 (1990). Cell lines containing the reporter gene fusions are then exposed to the agent to be tested under appropriate conditions and time. Differential expression of the reporter gene between samples exposed to the agent and control samples identifies agents that modulate the expression of a nucleic acid encoding AMP.

Additional assay formats may be used to monitor the ability of the agent to modulate the expression of a nucleic acid encoding AMP. For instance, mRNA expression may be monitored directly by hybridization to the nucleic acids of encoding AMP. Another way to evaluate AMP expression levels is to use either quantitative or semi-quantitative PCR. Semi-quantitative PCR is performed by using a thermo-stable DNA polymerase and temperature cycling to “amplify” a portion of a given cDNA species using specific oligonuceotide primers as described in Sambrook et al., Molecular Cloning: A Laboratory Manual, 2d edition, Cold Spring Harbor Press (1989). An example of quantitative PCR is the use of TaqMan™ analysis developed and described by Applied Biosystems, (ABI). Cell lines are exposed to the agent to be tested under appropriate conditions and time and total RNA or mRNA is isolated by standard procedures such those disclosed in Sambrook et al. Molecular Cloning—A Laboratory Manual, Cold Spring Harbor Laboratory Press (1989).

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementation that should exist between two nucleic acid strands in order to form a hybrid. Stringency should be chosen to maximize the difference in stability between the probe:target hybrid and probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al., Molecular Cloning—A Laboratory Manual, Cold Spring Harbor Laboratory Press, (1989) or Ausubel et al., Current Protocols in Molecular Biology, Greene Publishing Co. (1995).

Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The glass wafer can then be exposed to total cellular RNA or polyA RNA from a sample under conditions in which the affixed sequences will specifically hybridize. Such solid supports and hybridization methods are widely available, for example, those disclosed in WO 95/11755. By examining for the ability of a given probe to specifically hybridize to an RNA sample from an untreated cell population and from a cell population exposed to the agent, agents which up or down regulate the expression of a nucleic acid encoding HPTPbeta are identified.

Hybridization for qualitative and quantitative analysis of mRNA may also be carried out by using a RNase Protection Assay (i.e., RPA, see Ma et al., Methods 10, 273-238 (1996)). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes (pH 6.4), 0.4 M NaCl and 1 mM EDTA. The resulting hybrids are then digested in a buffer comprising 40 ug/ml ribonuclease A and 2 ug/ml ribonuclease. After deactivation and extraction of extraneous proteins, the samples are loaded onto urea/polyacrylamide gels for analysis.

In another assay format, cells or cell lines are first identified which express the gene products of AMP physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or other detectable marker. Such a process is well known in the art (see Sambrook et al., Molecular Cloning—A Laboratory Manual, Cold Spring Harbor Laboratory Press (1989)).

Cells or cell lines transduced or transfected as outlined above are then contacted with agents under appropriate conditions; for example, the agent in a pharmaceutically acceptable excipient is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modified as deemed necessary by one skilled in the art. Subsequent to contacting the cells with the agent, said cells are disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the agent-contacted sample compared to the control will be used to distinguish the effectiveness of the agent.

In one format, the specific activity of AMP is normalized to a standard unit, between a cell population that has been exposed to the agent to be tested and compared to an un-exposed control cell population. Cell lines or populations are exposed to the agent to be tested under appropriate conditions and time. Cellular lysates may be prepared from the exposed cell line or population and a control, unexposed cell line or population. The cellular lysates are then analyzed with a probe.

Antibody probes can be prepared by immunizing suitable mammalian hosts utilizing appropriate immunization protocols using the proteins of the invention or antigen-containing fragments thereof. While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, (see e.g., Kohler & Milstein, Biotechnology, 24, 524-526 (1992) or modifications which affect immortalization of lymphocytes or spleen cells, as is generally known.

Fragments of the monoclonal antibodies or the polyclonal antisera that contain the immunologically significant portion can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin, for instance, humanized antibodies. The antibody can therefore be a humanized antibody or human antibody, as described in U.S. Pat. No. 5,585,089 or Riechmann et al., Nature 332, 323-327 (1988).

One class of agents that may modulate AMP activity includes peptide mimetics that mimic the three-dimensional structure of an AMP protein. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others.

In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

In another form, peptide analogs are commonly used in the pharmaceutical industry as non-peptide drugs with properties analogous to those of the template peptide. These types of non-peptide compounds are also referred to as peptide mimetics or peptidomimetics, Fauchere, Adv. Drug Res., 15, 29-69 (1986); Veber & Freidinger, Trends Neurosci., 8, 392-396 (1985); Evans et al., J. Med. Chem., 30, 1229-1239 (1987) which are incorporated herein by reference and are usually developed with the aid of computerized molecular modeling.

Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage by methods known in the art.

Labeling of peptide mimetics usually involves covalent attachment of one or more labels, directly or through a spacer (e.g., an amide group), to non-interfering positions on the peptide mimetic that are predicted by quantitative structure-activity data and molecular modeling. Such non-interfering positions generally are positions that do not form direct contacts with the macromolecules to which the peptide mimetic binds to produce the therapeutic effect. Derivitization (e.g., labeling) of peptide mimetics should not substantially interfere with the desired biological or pharmacological activity of the peptide mimetic.

The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al., Proc. Natl. Acad. Sci., USA, 88, 9578-9582 (1991)) and using the phage display method. The two hybrid method detects protein-protein interactions in yeast, Fields et al., Nature, 340, 245-246 (1989). The phage display method detects the interaction between an immobilized protein and a protein that is expressed on the surface of phages such as lambda and M13, Amberg et al., Strategies, 6, 2-4 (1993); Hogrefe et al., Gene, 128, 119-126 (1993). These methods allow positive and negative selection for protein-protein interactions and the identification of the sequences that determine these interactions.

### IV. Method of Administrating an Agent that Modulates AMP Activity or Expression

One aspect of the invention provides for a method for preventing or treating an angiogenesis mediated disorder by administering a safe and effective amount of an agent that modulates AMP expression or AMP activity. Agents of the present invention may be administered by those methods well-known in the art.

For example, the delivery of antisense, triplex agents, ribozymes, competitive inhibitors and the like can be achieved using a recombinant expression vector such as a chimeric virus or a colloidal dispersion system. Various viral vectors which can be utilized for gene therapy as taught herein include, but are not limited to, adenovirus, herpes virus, vaccinia, or, in one embodiment, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting a polynucleotide sequence of interest into the viral vector, along with another gene that encodes the ligand for a receptor on a specific target cell, for example, the vector is now target specific. Preferred targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing the antisense polynucleotide.

Another targeted delivery system for antisense polynucleotides a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 um can encapsulate a substantial percentage of an aqueous buffer containing large macromolecules. RNA, DNA and intact virions can be encapsulated within the aqueous interior and be delivered to cells in a biologically active form, Fraley, et al., Trends Biochem. Sci., 6, 77 (1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information, Mannino, et al., Biotechniques, 6, 682, (1988).

These and other uses of antisense methods to inhibit the in vivo translation of genes are well known in the art (e.g., De Mesmaeker, et al., Backbone modifications in oligonucleotides and peptide nucleic acid systems, Curr. Opin. Struct. Biol., 5, 343-355 (1995); Gewirtz, A. M., et al., Facilitating delivery of antisense -oligodeoxynucleotides: Helping antisense deliver on its promise, Proc. Natl. Acad. Sci., U.S.A., 93, 3161-3163 (1996b); Stein, C. A., A discussion of G-tetrads, Exploiting the potential of antisense: beyond phosphorothioate oligodeoxynucleotides, Chem. and Biol., 3, 319-323 (1996).

### V. Diagnostic or Prognostic Methods

Expression of AMP may be used as a diagnostic marker for the prediction or identification of an angiogenesis mediated disorder. For example, a cell or tissue sample may be assayed for the expression levels of an AMP by any of the methods described herein and compared to the expression level found in normal health tissue. Such methods may be used to diagnose or identify angiogenesis mediated disorders.

Expression of AMP may also be used as a marker for monitoring that status, that is the progression, of an angiogenesis mediated disorder. Expression or activity of the AMP or nucleotides encoding the same may also used to track or predict the progress or efficacy of a treatment regime in a patient. For instance, a patient's progress or response to a given drug may be monitored by measuring gene expression of an AMP of the invention in a cell or tissue sample after treatment or administration of the drug. The expression of AMP in the post-treatment sample may then be compared to gene expression from the same patient before treatment.

### VI. Transgenic Animals

Transgenic animals containing mutant, knock-out or modified genes corresponding to AMP are also included in the invention. Transgenic animals are genetically modified animals into which recombinant, exogenous or cloned genetic material has been experimentally transferred. Such genetic material is often referred to as a transgene. The nucleic acid sequence of the transgene may be integrated either at a locus of a genome where that particular nucleic acid sequence is not otherwise normally found or at the normal locus for the transgene. The transgene may consist of nucleic acid sequences derived from the genome of the same species or of a different species than the species of the target animal.

The term “germ cell line transgenic animal” refers to a transgenic animal in which the genetic alteration or genetic information was introduced into a germ line cell, thereby conferring the ability of the transgenic animal to transfer the genetic information to offspring. If such offspring in fact possess some or all of that alteration or genetic information, then they too are transgenic animals.

The alteration or genetic information may be foreign to the species of animal to which the recipient belongs, foreign only to the particular individual recipient, or may be genetic information already possessed by the recipient. In the last case, the altered or introduced gene may be expressed differently than the native gene.

Transgenic animals can be produced by a variety of different methods including transfection, electroporation, microinjection, gene targeting in embryonic stem cells and recombinant viral and retroviral infection (see, e.g., U.S. Pat. Nos. 4,736,866 & 5,602,307; Mullins et al., Hypertension, 22, 630-633 (1993); Brenin et al., Surg. Oncol. 6, 99-110 (1997); Tuan, Recombinant Gene Expression Protocols, Methods in Molecular Biology, Humana Press (1997)).

A number of recombinant or transgenic mice have been produced, including those which express an activated oncogene sequence, U.S. Pat. No. 4,736,866; express simian SV40 T-antigen, U.S. Pat. No. 5,728,915; lack the expression of interferon regulatory factor 1 (IRF-1), U.S. Pat. No. 5,731,490; exhibit dopaminergic dysfunction, U.S. Pat. No. 5,723,719; express at least one human gene which participates in blood pressure control, U.S. Pat. No. 5,731,489; display greater similarity to the conditions existing in naturally occurring Alzheimer's disease, U.S. Pat. No. 5,720,936; have a reduced capacity to mediate cellular adhesion, U.S. Pat. No. 5,602,307; possess a bovine growth hormone gene, Clutter et al., Genetics, 143, 1753-1760 (1996); or are capable of generating a fully human antibody response, McCarthy, Lancet 349, 405-406 (1997).

While mice and rats remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (see, e.g., Kim et al., Mol. Reprod. Dev., 46, 515-526 (1997); Houdebine, Reprod. Nutr. Dev., 35, 609-617 (1995); Petters, Reprod. Fertil. Dev., 6, 643-645 (1994); Schnieke, et al., Science, 278, 2130-2133 (1997); and Amoah J., Animal Science, 75, 578-585 (1997)).

The method of introduction of nucleic acid fragments into recombination competent mammalian cells can be by any method that favors co-transformation of multiple nucleic acid molecules. Detailed procedures for producing transgenic animals are readily available to one skilled in the art, including the disclosures in U.S. Pat. Nos. 5,489,743 & 5,602,307.

### VII. Compositions

The compositions of the invention comprise: (a) a safe and effective amount of an AMP, or agent modulating AMP; and (b) a pharmaceutically-acceptable carrier. AMPs and said agents are formulated by standard pharmaceutical formulation techniques such as those disclosed in Remington's Pharmaceutical Sciences, Mack Publishing Company, Easton, Pa., latest edition.

A “safe and effective amount” of an AMP, or agent modulating AMP, is an amount that is effective, to either stimulate or inhibit angiongenesis, in an animal, preferably a mammal, more preferably a human subject, in need thereof, without undue adverse side effects (such as toxicity, irritation, or allergic response), commensurate with a reasonable benefit/risk ratio when used in the manner of this invention. The specific “safe and effective amount” will, obviously, vary with such factors as the particular condition being treated, the physical condition of the subject, the duration of treatment, the nature of concurrent therapy (if any), the specific dosage form to be used, the carrier employed, the solubility of the AMP or agent therein, and the dosage regimen desired for the composition. One skilled in the art may use the following teachings to determine a “safe and effective amount” in accordance with the present invention. Spilker B., Guide to Clinical Studies and Developing Protocols, Raven Press Books, Ltd., New York, 7-13, 54-60 (1984); Spilker B., Guide to Clinical Trials, Raven Press, Ltd., New York, 93-101 (1991); Craig C., and R. Stitzel, eds., Modem Pharmacology, 2d ed., Little, Brown and Co., Boston, 127-33 (1986); T. Speight, ed., Avery's Drug Treatment: Principles and Practice of Clinical Pharmacology and Therapeutics, 3d ed., Williams and Wilkins, Baltimore, 50-56 (1987); R. Tallarida, R. Raffa and P. McGonigle, Principles in General Pharmacology, Springer-Verlag, New York, 18-20 (1988).

In addition to the subject AMP or agent, the compositions of the subject invention contain a pharmaceutically-acceptable carrier. The term “pharmaceutically-acceptable carrier,” as used herein, means one or more compatible solid or liquid filler diluents or encapsulating substances which are suitable for administration to an animal, preferably a mammal, more preferably a human. The term “compatible”, as used herein, means that the components of the composition are capable of being commingled with the subject AMP or agent, and with each other respectively, in a manner such that there is no interaction that would substantially reduce the pharmaceutical efficacy of the composition under ordinary use situations. Pharmaceutically-acceptable carriers must, of course, be of sufficiently high purity and sufficiently low toxicity to render them suitable for administration to the animal, preferably a mammal, more preferably a human being treated.

Some examples of substances which can serve as pharmaceutically-acceptable carriers or components thereof are: sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tweens®; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the AMP or agent is to be administered.

If the subject AMP or agent is to be injected, the preferred pharmaceutically-acceptable carrier is sterile, physiological saline, with a blood-compatible colloidal suspending agent, the pH of which has been adjusted to about 7.4.

In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil. Preferably, the pharmaceutically-acceptable carrier, in compositions for parenteral administration, comprises at least about 90% by weight of the total composition.

The compositions of this invention are preferably provided in unit dosage form. As used herein, a “unit dosage form” is a composition of this invention containing an amount of an AMP or agent that is suitable for administration to an animal, preferably a mammal, more preferably a human subject, in a single dose, according to good medical practice. These compositions preferably contain from about 0.1 mg (milligrams) to about 1000 mg, more preferably from about 10 mg to about 500 mg, more preferably from about 10 mg to about 300 mg, of an AMP or agent.

The compositions of this invention may be in any of a variety of forms, suitable, for example, for oral, rectal, topical, nasal, ocular or parenteral administration. Depending upon the particular route of administration desired, a variety of pharmaceutically-acceptable carriers well-known in the art may be used. These include solid or liquid fillers, diluents, hydrotropes, surface-active agents, and encapsulating substances. Optional pharmaceutically-active materials may be included, which do not substantially interfere with the angiogenesis modulating activity of the AMP or agent of the invention. The amount of carrier employed in conjunction with the AMP or agent is sufficient to provide a practical quantity of material for administration per unit dose of the AMP or agent, respectively. Techniques and compositions for making dosage forms useful in the methods of this invention are described in the following references: Modern Pharmaceutics, Chapters 9 and 10 (Banker & Rhodes, editors, 1979); Lieberman et al., Pharmaceutical Dosage Forms: Tablets, (1981); and Ansel, Introduction to Pharmaceutical Dosage Forms, 2d Edition (1976).

Various oral dosage forms can be used, including such solid forms as tablets, capsules, granules and bulk powders. These oral forms comprise a safe and effective amount, usually at least about 5%, and preferably from about 25% to about 50%, of AMP. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, and containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of the subject invention, and can be readily made by a person skilled in the art. In general, the formulation will include the protein (or chemically modified protein), and inert ingredients which allow for protection against the stomach environment, and release of the biologically active material in the intestine.

The AMP may be chemically modified so that oral delivery of the derivative is efficacious. Generally, the chemical modification contemplated is the attachment of at least one moiety to the protein molecule itself, where said moiety permits (a) inhibition of proteolysis; and (b) uptake into the blood stream from the stomach or intestine. Also desired is the increase in overall stability of the protein and increase in circulation time in the body. Examples of such moieties include: polyethylene glycol, copolymers of ethylene glycol and propylene glycol, carboxymethyl cellulose, dextran, polyvinyl alcohol, polyvinyl pyitolidone and polyproline, Abuchowski et al., supra (1981); Newmark et al., J. Appl. Biochem., 4:185-189 (1982). Other polymers that could be used are poly-1,3-dioxolane and poly-1,3,6-tioxocane. Preferred for pharmaceutical usage, as indicated above, are polyethylene glycol moieties.

For the AMP, the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the protein (or derivative) or by release of the biologically active material beyond the stomach environment, such as in the intestine.

To ensure full gastric resistance, a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel® RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit® coatings, waxes and shellac.

Compositions of the subject invention may optionally include other active agents. Non-limiting examples of active agents are listed in WO 99/15210.

Other compositions useful for attaining systemic delivery of the subject compounds include sublingual, buccal, suppository, and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compositions of this invention can also be administered topically to a subject, e.g., by the direct laying on or spreading of the composition on the epidermal or epithelial tissue of the subject, or transdermally via a “patch.” An example of a suitable patch applicator is described in U.S. patent application Ser. No. 10/054,113. Such compositions include, for example, lotions, creams, solutions, gels and solids. These topical compositions preferably comprise a safe and effective amount, usually at least about 0.1%, and preferably from about 1% to about 5%, of the AMP. Suitable carriers for topical administration preferably remain in place on the skin as a continuous film, and resist being removed by perspiration or immersion in water. Generally, the carrier is organic in nature and capable of having dispersed or dissolved therein the AMP. The carrier may include pharmaceutically-acceptable emollients, emulsifiers, thickening agents, solvents and the like.

### VIII. Methods of Administration

This invention also provides methods of treating angiogenesis elevated disorders or angiogenesis reduced disorders in a human or other animal subject, by administering a safe and effective amount of an AMP or agent modulating AMP to said subject. The methods of the invention are useful in treating or preventing disorders described above.

Compositions of this invention can be administered topically or systemically. Systemic application includes any method of introducing AMP or agent into the tissues of the body, e.g., intra-articular (especially in treatment of rheumatoid arthritis), intrathecal, epidural, intramuscular, transdermal, intravenous, intraperitoneal, subcutaneous, sublingual, rectal, and oral administration.

The specific dosage of AMP or agent to be administered, as well as the duration of treatment, and whether the treatment is topical or systemic are interdependent. The dosage and treatment regimen will also depend upon such factors as the specific AMP or agent used, the treatment indication, the ability of the AMP or agent to reach minimum inhibitory concentrations at the site of the tissue in need of treatment, the personal attributes of the subject (such as weight), compliance with the treatment regimen, and the presence and severity of any side effects of the treatment.

Typically, for a human adult (weighing approximately 70 kilograms), from about 1 mg to about 3000 mg, more preferably from about 5 mg to about 1000 mg, more preferably from about 10 mg to about 100 mg, of AMP or agent are administered per day for systemic administration. It is understood that these dosage ranges are by way of example only, and that daily administration can be adjusted depending on the factors listed above.

Topical administration can be used to deliver the AMP or agent systemically, or to treat a subject locally. The amounts of AMP or agent to be topically administered depends upon such factors as skin sensitivity, type and location of the tissue to be treated, the composition and carrier (if any) to be administered, the particular AMP or agent to be administered, as well as the particular disorder to be treated and the extent to which systemic (as distinguished from local) effects are desired.

For localized conditions, topical administration is preferred. For example, to treat a retinal/choroidal neovascularization disease, direct application to the affected eye may employ a formulation as eyedrops or aerosol. For corneal treatment, the compounds of the invention can also be formulated as gels, drops or ointments, or can be incorporated into collagen or a hydrophilic polymer shield. The materials can also be inserted as a contact lens or reservoir or as a subconjunctival formulation. For treatment of a skin disease, the AMP or agent is applied locally and topically, in a gel, paste, salve or ointment. For treatment of oral diseases, the AMP or agent may be applied locally in a gel, paste, mouth wash, or implant. The mode of treatment thus reflects the nature of the condition and suitable formulations for any selected route are available in the art.

The AMP or agent of the present invention can be targeted to specific locations where treatment is needed by using targeting ligands. For example, to focus AMP or agent to inhibit angiogenesis in a tumor, the AMP or agent is conjugated to an antibody or fragment thereof which is immunoreactive with a tumor marker as is generally understood in the preparation of immunotoxins in general. The targeting ligand can also be a ligand suitable for a receptor which is present on the tumor. Any targeting ligand which specifically reacts with a marker for the intended target tissue can be used. Methods for coupling the invention compound to the targeting ligand are well known and are similar to those described below for coupling to carrier. The conjugates are formulated and administered as described above.

AMP or agent may be administered via a controlled release. For example, the AMP or agent may be administered using intravenous infusion, an implantable osmotic pump, a transdermal patch, liposomes, or other modes of administration. In one embodiment, a pump may be used, Langer et al., eds., Medical Applications of Controlled Release, CRC Pres., Boca Raton, Fla. (1974); Sefton, CRC Crit. Ref. Biomed. Eng., 14, 201 (1987); Buchwald et al., Surgery, 88, 507 (1980); Saudek et al., N. Engl. J. Med., 321, 574 (1989). In another embodiment, polymeric materials can be used (see, Langer, supra (1974); Sefton, supra (1987); Smolen et al., eds., Controlled Drug Bioavailability, Drug Product Design and Performance, Wiley, N.Y. (1984); Ranger et al., J. Macromol. Sci. Rev. Macromol. Chem., 23, 61 (1983); see also Levy et al., Science, 228, 190 (1985); During et al., Ann. Neurol., 25, 351 (1989); Howard et al., J. Neurosurg., 71, 105 (1989)). In yet another embodiment, a controlled release system can be placed in proximity of the therapeutic target thus requiring only a fraction of the systemic dose (see. e.g., Goodson, in Medical Applications of Controlled Release, 2, 115-138 (1984)). Other controlled release systems are discussed in the review by Langer, Science, 249, 1527-1533 (1990). In another embodiment, the therapeutic compound can be delivered in a vesicle, in particular a liposome (see Langer, 1990, supra); Treat et al., in Liposomes in the Therapy of Infectious Disease and Cancer, Lopez-Berestein and Fidler (eds.), Liss, New York, 353-365 (1989); Lopez-Berestein, 317-327; see generally ibid.).

In all of the foregoing, of course, the AMPs or agents of the invention can be administered alone or as mixtures, and the compositions may further include additional drugs or excipients as appropriate for the indication.

### IX. Kits

The present invention includes a kit for preventing or treating an angiogenesis mediated disorder comprising: (a) an AMP or agent modulating AMP in a unit dose form; (b) usage instructions; and (c) optionally a package containing components (a) and (b). Such a kit preferably includes a number of unit dosages. Such kits can include a card having dosages oriented in the order of their intended use. An example of such a kit is a “blister pack.” Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered. A non-limiting example of a kit is described in WO 01/45636. Treatments schedules are within the purview of those skilled in the medicinal arts.

### EXAMPLES

### 1. Introduction

To better understand the molecular mechanisms of angiogenesis, we utilized a proteomic approach to try to improve our understanding of this complex process.

The rat cornea model of angiogenesis is one of the most extensively studied and widely accepted models of blood vessel growth, McCracken, J. S. et al., Lab. Invest., 41, 519-530 (1979); Klintworth, G. K., Int. Ophthalmol. Clin., 23, 27-39 (1981); Kenyon, K. R., The Cornea. Scientific Foundations and Clinical Practice, Little, Brown and Co., Boston, 63-98 (1987); Burger, P. C. et al., Lab. Invest., 45, 328-335 (1981); Burger, P. C. et al., Lab. Invest., 48, 169-180 (1983); Ross, L. L. et al., Exp. Eye Res., 61, 435-450 (1995). This model is attractive to researchers because it provides an in vivo environment in which to study this complex process with convenient access to the corneal tissue and the highly visible, developing vasculature, Klintworth, G. K. et al., Int. Ophthalmol. Clin., 23, 27-39 (1983). The rat cornea model of angiogenesis was selected for our proteome analysis for three reasons. First, the cornea is normally an avascular tissue that can be stimulated to undergo angiogenesis in response to silver nitrate cauterization, Ross, L. L. et al., Exp. Eye Res., 61, 435-450 (1995); Haynes, W. L. et al., Invest. Ophthalmol. Vis. Sci., 30, 1588-1593 (1989); Scroggs, M. W. et al., Invest. Ophthalmol. Vis. Sci., 32, 2105-2111 (1991). Therefore, we can compare protein expression profiles between angiogenic and non-angiogenic tissues to detect changes in protein expression. Second, the time course of angiogenesis following cauterization is highly reproducible and well characterized so that we can examine changes in protein expression at all stages of blood vessel formation, from the initial vascular sprouting through smooth muscle recruitment and vessel maturation, Proia, A.D. et al, Lab. Invest., 58, 473-479 (1988). Lastly, an in vivo model like this provides a more physiologic approach to angiogenesis than the available in vitro models.

We present the protein expression profiles from the rat cornea at various stages of angiogenesis. We describe two distinct patterns of protein expression during the time course of blood vessel formation. Lastly, we identify proteins that are differentially expressed during angiogenesis by matrix-assisted laser desorption ionization time-of-flight and high pressure liquid chromatography-coupled electrospray ionization tandem mass spectrometry.

### 2. Materials and Methods

2.1. Rat Cornea Model of Angiogenesis

30 Zivic-Miller Sprague-Dawley rats (200-224 gram females) were used for these studies. Rats were anesthetized using diethyl ether and given 0.1 mg of butorphanol intraperitoneally. The right eye of each animal was cauterized by pressing an applicator stick coated with 75% silver nitrate/25% potassium nitrate (Graham-Field Surgical Co., Inc., New Hyde Park, N.Y.) to the center of the cornea for 5 seconds to stimulate angiogenesis as previously described in Ross, L. L. et al., Exp. Eye Res., 61, 435-450 (1995); Haynes, W. L. et al., Invest. Ophthalmol. Vis. Sci., 30, 1588-1593 (1989); Scroggs, M. W. et al., Invest. Ophthalmol. Vis. Sci., 32, 2105-2111 (1991), while the untreated, left eye of each animal served as a control. At each time point (days 2, 3, 4, 6, 7 and 15 post-cautery), 5 rats were euthanized by asphyxiation in a carbon dioxide chamber. Both eyes of each animal were enucleated and the corneoscleral rims removed as previously described in Ross, L. L. et al. Exp. Eye Res., 61, 435-450 (1995); Haynes, W. L. et al., Invest. Ophthalmol. Vis. Sci., 30, 1588-1593 (1989); Scroggs, M. W. et al Invest. Ophthalmol. Vis. Sci., 32, 2105-2111 (1991). Corneas were immediately frozen at −80° C. for 2-dimensional electrophoresis analysis. In a parallel experiment at days 0, 2, 4, 7, and 15 post-cautery, rats were sacrificed and perfused with 10-20 ml of a mixture of 10% Higgins Drawing Ink (Faber-Castell Corp., Newark, N.J.) mixed with 11% gelatin in lactated Ringer's solution prior to harvesting corneas to visualize the vasculature as previously described in Haynes, W. L. et al., Invest. Ophthalmol. Vis. Sci., 30, 1588-1593 (1989); Scroggs, M. W. et al., Invest. Ophthalmol. Vis. Sci., 32, 2105-2111 (1991). Corneal flat preparations were prepared and mounted on microscope slides for imaging.

2.2. Sample Preparation and 2-Dimensional Gel Electrophoresis

Corneas were individually solubilized in 50 microliters of sample buffer (9M urea, 2% CHAPS, 2% DTT, 0.1% SDS, trace bromophenol blue, and 0.5 tablet/ml of Complete protease inhibitor cocktail tablet (Roche Molecular Biochemicals, Indianapolis, Ind.)) by grinding them in a liquid nitrogen-chilled mortar and pestle. Protein extracts were cleared by centrifugation at 14,000 rpm for 5 minutes. Sample volumes were increased to 400 μl with isoelectric focusing buffer (9M urea, 2% CHAPS, 2% dithiothreitol, and 1% Pharmalyte, pH 3-10 (Amersham Pharmacia Biotech, Piscataway, N.J.)). Samples were loaded overnight onto 18 cm Immobiline Drystrip gels, pH 4-7 (Amersham Pharmacia Biotech, Piscataway, N.J.) and resolved by isoelectric focusing at 3,500 volts for 15 hours. Samples strips were then equilibrated for 30 minutes in Tris Acetate Equilibration buffer (Genomic,Solutions, Ann Arbor, Mich.) supplemented with 1% sodium dodecyl sulfate and 0.77% dithiothreitol. Following equilibration, strips were loaded onto 10% tricine gels (Genomic Solutions, Ann Arbor, Mich.) and resolved in the second dimension at 1100 milliwatts per gel at 4° C. Proteins on gels were visualized using the Investigator Silver Stain kit (Genomic Solutions, Ann Arbor, Mich.) according to the manufacturer except that gluteraldehyde was omitted from the fixation step since this crosslinking reagent interferes with extraction of the proteins for identification.

2.3. Image Analysis and Protein Identification

Silver stained 2-DE gels were scanned using a Personal Densitometer SI (Amersham Pharmacia Biotech, Piscataway, N.J.). The digitized protein expression profiles at each time point post-cautery were compared in triplicate to controls using the Z3 2D gel image analysis software (Compugen, Jamesburg, N.J.). Differentially-expressed proteins that varied greater than 2-fold in level of expression were excised from the gels and destained in a solution of freshly prepared 50 mM sodium thiosulfate and 15 mM potassium ferricyanide as previously described in Gharahdaghi, F. et al., Electrophoresis, 20, 601-605 (1999). Proteins were then digested with 20 ng of sequencing grade porcine trypsin (Promega, Madison, Wis.) in 50 mM ammonium bicarbonate overnight to generate tryptic peptides. The peptide digests were recovered by 100 microliters of 60% acetonitrile, 0.1% trifluroacetic acid solution. Shevchenko, A. et al., Anal. Chem., 68, 850-858 (1996). Samples were dried extensively in a speedvac to remove residual ammonium bicarbonate. Peptides were resuspended in 5 μl of a 50% acetonitrile/0.3% trifluoroacetic acid solution. 0.75 μl (15%) of each sample was combined with 1.5 μl of matrix solution (10 mg/ml α-cyano-4-hydroxycinnamic acid, 60% acetonitrile, and 0.3% trifluoroacetic acid) and analyzed by matrix-assisted laser desorption ionization time-of-flight mass spectrometry. Spectra were collected on a Voyager DE-STR matrix-assisted laser desorption ionization time-of-flight mass spectrometer (Applied Biosystems, Framingham, Mass.) in positive ion, reflector mode with delayed ion extraction using the following conditions: nitrogen laser at 337 nm; accelerating voltage at 20 kV; grid voltage at 75%; ion delay of 150 nsec and a mass range of 800-3200 Da. Internal mass calibration was performed using peptides arising from auto-digestion of the porcine trypsin. The MS-Fit algorithm from the ProteinProspector (v3.4.1) search engine (http://prospector.ucsf.edu/) purchased from University of California-San Francisco was used for protein identification, Clauser, K. R. et al., Anal. Chem., 71, 2871-2878 (1999). In the event that the protein identification was ambiguous after the matrix-assisted laser desorption ionization time-of-flight step, the remaining 85% of the peptide in each sample was analyzed by capillary liquid chromatography electrospray ionization tandem mass spectrometry with an LCPackings Ultimate capillary liquid chromatography system equipped with a FAMOS micro-autosampler and a 5 cm×300 μm i.d. PepMap C18 column (LCPackings, San Francisco, Calif.) coupled to a Finnigan LCQ^{Deca} ion trap mass spectrometer (ThermoFinnigan, San Jose, Calif.). Data were collected and analyzed using Finnigan Xcalibur v. 1.2 software in data-dependent scan mode such that any peptide signal over 1×10⁵ intensity triggered the automated acquisition of a tandem mass spectrometry fragmentation spectrum for that peptide. The collective tandem mass spectrometry spectra for each capillary liquid chromatography-coupled tandem mass spectrometry run were searched against the National Center for Biotechnology Information nr database using Mascot Daemon (v. 1.7.1) as a client attached to the Mascot search protocol (Matrix Science, Ltd.; http://www.matrixscience.com) Perkins, et al., Electrophoresis, 20, 3551-3567 (1999).

### 3. Results

3.1. Progression of Angiogenesis in the Rat Cornea Model.

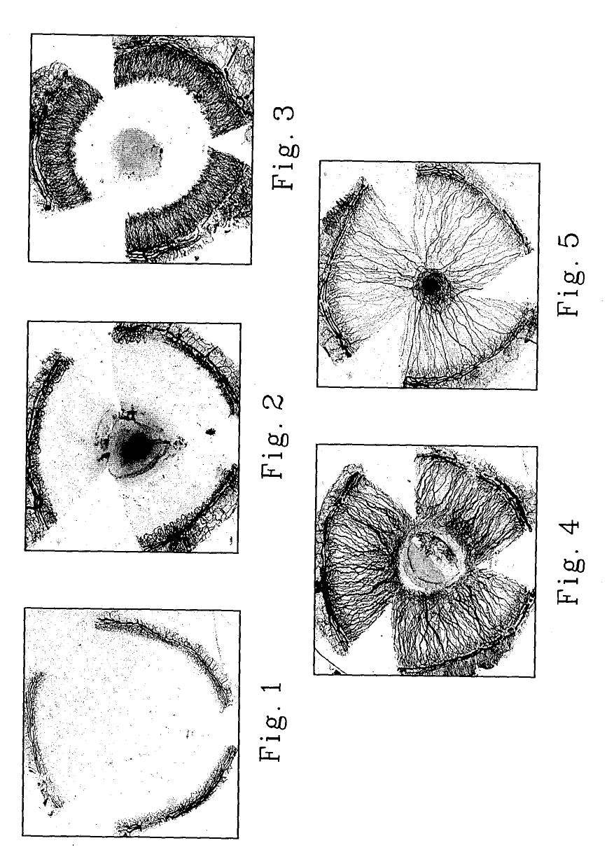

Silver nitrate cauterization of the rat eye has previously been used to promote angiogenesis in the normally avascular cornea, Ross, L. L. et al., Exp. Eye Res., 61, 435-450 (1995); Haynes, W. L. et al., Invest. Ophthalmol. Vis. Sci., 30, 1588-1593 (1989); Scroggs, M. W. et al., Invest. Ophthalmol. Vis. Sci., 32, 2105-2111 (1991); Proia, A. D. et al., Lab. Invest., 58, 473-479 (1988). This method produces a discrete, central lesion on the cornea resulting in necrosis of the corneal epithelium and stroma. In the day 0, non-cauterized cornea, only the limbal vessels at the periphery of the cornea are seen (FIG. 1.) In just 24 hours after the injury, these limbal vessels appear slightly engorged. Within 48 hours the limbal arcades are extended measurably further into the cornea with numerous short vascular sprouts projecting centrally toward the site of injury (FIG. 2, Day 2). By 3 and 4 days post cautery, this dense brushwork of vessels elongates evenly into the cornea from all sides (FIG. 3). Regression and remodeling of these new blood vessels occurs between days 6 and 7 as redundant vessels are “pruned” (FIG. 4) until only a few stable, mature vessels remain in the cornea (FIG. 5). Identification of the differentially-expressed proteins in this corneal model during the various stages of blood vessel growth will likely lead to a better understanding of the process of angiogenesis.

3.2 Differential Protein Expression

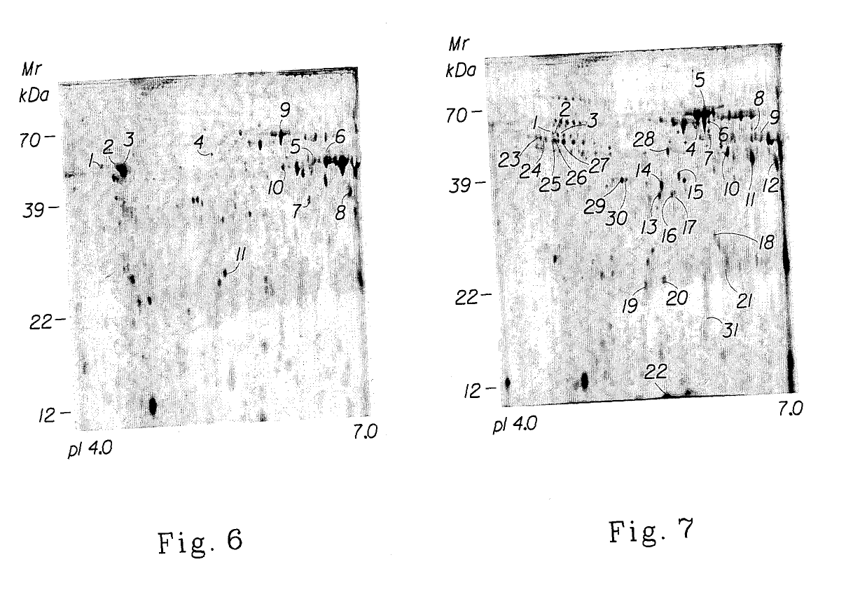

As this is the first reported application of proteome analysis on corneal tissue, several of the conditions for sample preparation and solubilization were developed de novo. We found that our protocol (described in Materials and methods) consistently generates enough protein from a single rat cornea to be visualized by 2-dimensional electrophoresis. To determine if our approach would be sensitive enough to detect differential protein expression using this model, we first examined protein expression profiles in angiogenic corneas at day 3 post-cautery (FIG. 7). This time point was selected because corneas at this time point are known to contain abundant blood vessel growth (see FIG. 3). These protein expression profiles were compared to those of non-cauterized controls (FIG. 6) to look for changes in protein expression. In this initial study, 11 spots were detected and identified that were down-regulated in response to silver nitrate cauterization/angiogenesis, while 31 spots were identified that were upregulated after cauterization as compared to control corneal proteins. These 42 spots accounted for 19 distinct proteins, as some of the spots were modified versions of the same protein (Table II). Therefore, this model is sufficient for proteome analysis as changes in corneal protein expression can be visualized by 2-dimensional electrophoresis and differentially-expressed proteins can be identified by Matrix-assisted laser desorption ionization time-of-flight mass spectrometry.

3.3 Differential Protein Expression in All Stages of Angiogenesis.

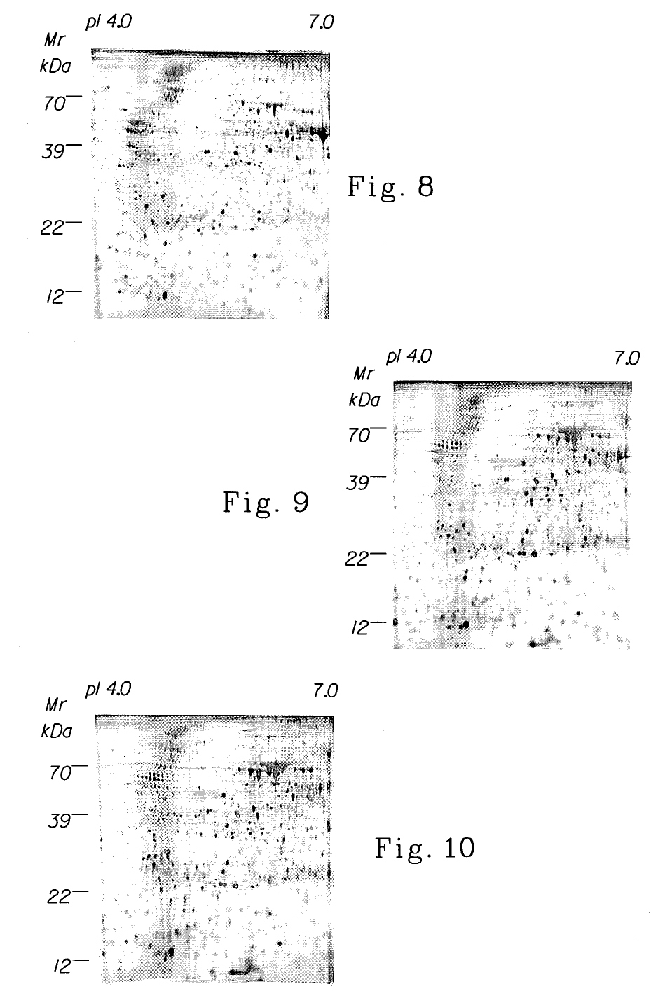

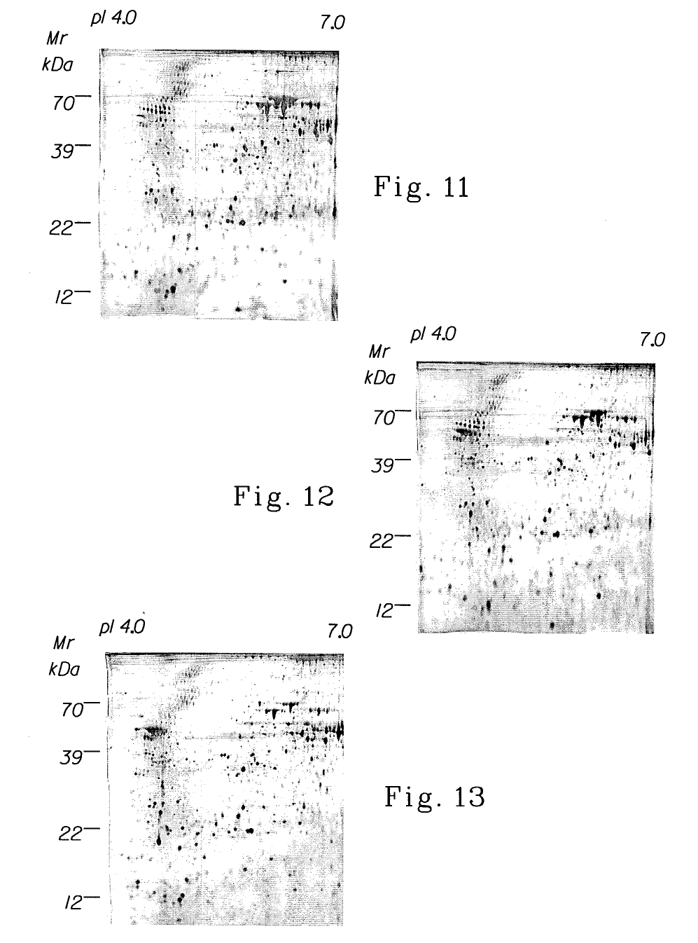

Analysis of the rat cornea proteome provides a “snapshot” of the cellular proteins expressed at any given time. However, to fully characterize and analyze a dynamic process like angiogenesis, experiments should be performed that capture the protein expression profiles of the cornea at the various stages of blood vessel development. Therefore, we designed a time course experiment to examine the changes in protein expression at the critical time points throughout the entire angiogenic process. Corneas were harvested in triplicate at day 0 and at days 0, 2, 4, 6, 7, and 15 post-cautery and their protein expression profiles were visualized by 2-dimensional electrophoresis (FIG. 8-13, respectively).

Protein spots that changed greater than 2-fold in level of expression (relative to control) throughout the time course were excised for identification. 101 spots were successfully identified from this time course experiment. However, several of these spots represented modified versions or fragments of the same protein. For example, 19 of the proteins were found to be fragments of collagen. This is not surprising when one considers that the cornea consists of an anterior squamous epithelium of 5-7 cell layers overlying a relatively thick extracellular stroma containing ordered collagen fibers, Xu, J. et al., J. Biol. Chem., 275, 24645-24652 (2000). In fact, these multiple fragments of collagen might be indicative of matrix metalloproteinase activities, which are already implicated during angiogenesis, Xu, J. et al., J. Cell Biol., 54, 1069-1079 (2001); Pepper, M. S., Arterioscler. Thromb. Vasc. Biol., 21, 1104-1117 (2001); John, A. et al., Pathol. Oncol. Res., 7, 14-23 (2001). A total of 48 distinct, differentially-expressed proteins were identified in this model of angiogenesis. These proteins are grouped according to their patterns of expression and listed in Table III.

### 4. Discussion

The term proteome, coined just a few years ago, Wasinger, V. C. et al., Electrophoresis, 16, 1090-1094 (1995), refers to all proteins present in a cell, tissue, or organism at any given time, including modified proteins arising from alternatively sliced transcripts or posttranslational modifications, Arrell, D. K. et al., Circ. Res., 88, 763-773 (2001). Since proteins are involved in nearly every cellular process, control every regulatory mechanism, become modified in disease (as the cause or effect), and provide targets for most drug treatments, Arrell, D. K et al., Circ. Res., 88, 763-773 (2001), it is imperative that we look to the proteome for a better understanding of a complex process like angiogenesis.

Angiogenesis research has traditionally focused on studies of gene regulation at the level of mRNA expression (i.e. the transcriptome). However, Anderson et at. have demonstrated that there is generally poor correlation between mRNA and cognate protein expression levels, Anderson, L. et al., Electrophoresis, 18, 533-537 (1997). In addition, many cellular processes are typically regulated through posttranslational modifications of proteins (e.g. phosphorylation), which would likely go undetected using solely a genomic approach. Therefore we utilized a proteomic approach in our studies to provide a better understanding of the process of angiogenesis in the rat cornea model.

In our proteome analysis of the rat cornea model, we identified more than 100 proteins that changed in expression level in response to silver nitrate cauterization/angiogenesis. These differentially-expressed proteins can be categorized into two basic groups: proteins that are down-regulated in response to silver nitrate cauterization/angiogenesis or proteins that become up-regulated. We might expect to find some of the down-regulated proteins to be natural inhibitors of angiogenesis. Hence, their down-regulation might enable/signal blood vessel growth to proceed. Similarly, we would predict some of the up-regulated proteins to be positive regulators of angiogenesis, blood vessel structural proteins, or blood components. Therefore, a comprehensive list of up-regulated proteins should include pro-angiogenic factors, matrix metalloproteinases, smooth muscle components, and important intracellular signaling molecules for endothelial cell migration and proliferation.

A majority of the proteins that we successfully identified are highly-abundant, proteins. In fact, several of these proteins are commonly seen in other studies of cardiovascular proteomics, Arrell, D. K. et al., Circ. Res., 88, 763-773 (2001). This is not surprising when one considers that current 2-dimensional electrophoresis and mass spectrometry technology in proteomics is somewhat limiting as less abundant, membrane-associated, and high molecular weight proteins are difficult to analyze, Washburn, M. P. et al., Curr. Opin. Microbiol., 3, 292-297 (2000); Gygi, S. P. et al., Proc. Natl. Acad. Sci. USA, 97, 9390-9395 (2000). Therefore, housekeeping proteins (>10,000 copies per cell), which constitute a significant portion of the cellular proteome, Blackstock, et al., Trends Biotech., 17, 121-127 (1999), are more frequently identified.

A notable trend in protein expression was observed in which a subset of proteins demonstrated a dramatic up-regulation by day 2 post-cautery, a peak in expression around day 6 or 7, and then a steady decline by day 15. The proteins that followed this pattern of expression were identified as hemopexin, serotransferrin, \tilde{α}1-macroglobulin, apolipoprotein(s), and albumin(s). Interestingly, all of these are predominantly blood proteins. This is not surprising when you consider that the cornea is transformed from an avascular tissue to a highly vascularized one, which would appear as an apparent up-regulation of these blood proteins. In the regression stages of angiogenesis as redundant vessels are resorbed (between days 7 and 15), the cornea becomes less highly vascularized which would explain the relative down-regulation of these proteins. This same pattern of expression would also be expected for structural proteins of these newly formed blood vessels. These findings validate ongoing efforts in proteomics in that they demonstrate that physiological changes can be detected at the level of the proteome.

In addition to the blood, structural, and house-keeping proteins, we also identified a number of other interesting proteins. Kininogen, phosphatidyl ethanolamine binding protein, heat shock protein 27, gelsolin, lipocortin I, lipocortin II, and RHO-GDP dissociation inhibitor-2 were all differentially expressed during the process of angiogenesis. Many of these can be linked to cellular proliferation and migration events that are required for angiogenesis and vascular remodeling, Colman, R. W., Biol. Chem., 382, .65-70 (2001); Hengst, U. et al., J. Biol. Chem., 276, 535-540 (2001); D'Amico, M. et al., FASEB J., 14, 1867-1869 (2000); Winston, J. S. et al., Breast Cancer Res. Treat., 65, 11-21 (2001); Hansen, R. K. et al., Biochem. Biophys. Res. Commun., 282, 186-193 (2001); Adra, C. N. et al., Genes Chromosomes Cancer, 8, 253-261 (1993). Therefore These proteins may importantly regulate the cellular events required for blood vessel assembly.

The rat cornea model of angiogenesis has been used previously to look for changes in gene expression in response to silver nitrate cauterization for the identification of important wound healing genes at early time points post-cautery, Ross, L. L. et al., Exp. Eye Res., 61, 435-450 (1995). A subtractive hybridization approach was used to identify 76 clones in the rat cornea whose corresponding mRNA levels increased in response to cauterization, Ross, L. L. et al., Exp. Eye Res., 61, 435-450 (1995). Of these clones, only aldehyde dehydrogenase and alpha crystallin were detected by our proteomic approach. However, in our studies the expression level of these proteins actually decreased in response to cauterization even though their cognate mRNAs were reported to increase. One potential explanation for our observation is that the decrease in expression of these proteins triggers a corresponding increase in gene transcription (and mRNA levels) to restore the protein levels to their pre-cauterized states. Regardless of the explanation, this lack of correlation underscores the importance of using a combination of both genomic and proteomic approaches to study complex biological systems like angiogenesis.

TABLE II

| Down-regulated proteins: | | Up-regulated proteins: | |
|---|---|---|---|
| Spot # | Protein ID | Spot # | Protein ID |
| 1 | calreticulin | 1, 2, 3 | serine protease |
| | precursor (CRP55) | | inhibitor 1 |
| 2, 3 | Keratin complex I, | 4, 6 | Hemopexin precursor |
| | acidic | | |
| 4 | Heat shock protein | 7, 10-18, 21, | Serum albumin |
| | 60 precursor | 28, 31 | precursor |
| 5, 6, | Aldehyde | 8, 9 | Class 3 aldehyde |
| 10 | dehydrogenase | | dehydrogenase |
| | class 3 | | complex with NAD |
| 7 | Capping protein | 19 | Myosin heavy chain, |
| | (actin filament) | | smooth muscle |
| 8 | Alcohol dehydro- | 20 | Apolipoprotein A-i |
| | genase, class IV | | |
| 9 | Heat shock prtein 70 | 22 | Calgranulin A |
| 11 | Ribosomal protein | 23, 24, 26 | Kallikrein-binding |
| | S6 kinase | | protein precursor |
| | | 25, 27 | Alpha-2-HS- |
| | | | glycoprotein |
| | | 29, 30 | beta actin |

TABLE III
Proteins that decrease post-cautery and remain lower than control levels:

| | |
|---|---|
| pro-collagen, type VII, αl | Aldehyde dehydrogenase, class 3 |
| (2 spots) | |
| collagen, α3 (VI) (5 spots) | beta actin |

Proteins that decrease post-cautery and then rebound:

| | |
|---|---|
| heat shock protein 70 | aldehyde dehydrogenase, class 3 (4 spots) |
| reticulocalbin (2 spots) | phosphatidyl ethanolamine binding protein |
| FIP2 | collagen, αl, type VI |
| macrophage capping protein | heat shock protein 27 |
| collagen, α3 (VI) | alpha crystallin A chain |
| heat shock protein 60 | |

Proteins that increase post-cautery and then remain higher than
control levels:

| | |
|---|---|
| gelsolin precursor | tropomyosin (2 spots) |
| serum albumin precursor | transthyretin precursor (pre-albumin) |
| (4 spots) | (2 spots) |
| beta actin (3 spots) | α-tropomyosin |
| cystatin beta | vimentin (3 spots) |
| vitamin D binding protein | fatty acid binding protein |
| precursor | |
| tropomyosin 4 | eukaryotic translation elongation factor |

Proteins that increase post-cautery, peak day 6-7, and then decline:

| | |
|---|---|
| gelsolin precursor | tropomyosin (2 spots) |
| t-kininogen (2 spots) | hemopexin precursor (2 spots) |
| serum albumin precursor | apolipoprotein C-IV |
| (14 spots) | |
| apolipoprotein A-IV precursor | pyruvate kinase (3 spots) |
| preprohaptoglobin (3 spots) | collagen α3 (VI) (2 spots) |
| galectin 7 | alpha-1-macroglobulin (3 spots) |
| lipocortin-III (annexin-III) | 40S ribosomal protein P40 |
| beta actin (5 spots) | alpha-2U-globulin precursor |
| serotransferrin precursor | lipocortin-I |
| apolipoprotein AI | phosphoglycerate kinase |
| apolipoprotein AI precursor | RHO GDP-dissociation inhibitor 2 |
| collagen α3 (VI) | Fab fragment of a rat antibody |
| malate dehydrogenase | calgranulin A |
| thioredoxin | |

### X. Miscellaneous

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

All documents cited are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A method of treating an angiogenesis-mediated disorder in a subject in need thereof by administering a therapeutically effective amount of SEQ ID NOS 1-308 or a variant thereof.

2) The method of claim 1, wherein SEQ ID NO selected from the group consisting of 2, 4, 6, 8, 10, 12, 14, 16, 18, 20, 22, 24, 26, 28, 30, 32, 34, 36, 38, 40, 42, 44, 46, 48-80, 82, 84, 86, 88, 90, 92, 93, 95, 97, 99, 101, 103, 150, 107, 109, 111, 113, 115-133, 135-153, 155, 157, 159, 161, 163, 165, 167, 169-172, 174, 176, 178, 180, 182, 184-185, 187, 189, 191, 193, 194, 196, 197, 199, 201, 203, 205, 207, 209-211, 213, 215, 217, 219-236, 238, 285, 287, 289, 291, 293, 295, 297, 298, 300, 302, 304, 306 and 308.

3) The method of claim 1, wherein the angiogenesis mediated disorder comprises an angiogenesis elevated disorder.

4) The method of claim 3, wherein the angiogenesis elevated disorder is selected from diseases associated with retinal or choroidal neovascularization, and diseases associated with chronic inflammation.

5) The method of claim 1, wherein the angiogenesis mediated disorder comprises an angiogenesis reduced disorder.

6) The method claim 5, wherein the angiogenesis reduced disorder is selected from the group consisting of myocardial ischema, stroke, coronary artery disease, and peripheral vascular disease.

7) A method of screening an agent useful for treating an angiogenesis mediated disorder comprising the steps of: (a) exposing an angiogenesis modulating protein to the agent; and (b) measuring activity of angiogenesis modulating protein; wherein a modulation in the angiogenesis modulating protein activity indicates the agent is useful for treating the angiogenesis mediated disorder.

8) A method of screening an agent useful for treating an angiogenesis mediated disorder comprising the steps of: (a) exposing an angiogenesis modulating protein to the agent; (b) measuring binding of the agent to the angiogenesis modulating protein; wherein binding of the agent to the angiogenesis modulating protein indicates the agent is useful for treating the angiogenesis mediated disorder.

9) A method of screening an agent useful for treating an angiogenesis mediated disorder comprising the steps of: (a) exposing an angiogenesis modulating protein encoding nucleotide to the agent; (b) measuring the binding of the agent to the angiogenesis modulating protein encoding nucleotide; wherein binding of the agent to the angiogenesis modulating protein encoding nucleotide indicates the agent is useful for treating the angiogenesis mediated disorder.

10) A method of screening an agent useful for treating an angiogenesis mediated disorder comprising the steps of: (a) exposing a cell to the agent; and (b) measuring expression or activity of angiogenesis modulating protein in the cell; wherein a modulation in the expression or the activity of angiogenesis modulating protein indicates the agent is useful for the treatment of the angiogenesis mediated disorder.

11) A method of screening an agent useful for treating an angiogenesis mediated disorder comprising the steps of: (a) exposing a cell to the agent; and (b) measuring the association of the agent and angiogenesis modulating protein; wherein a modulation in the association of the agent and angiogenesis modulating protein indicates the agent is useful for the treatment of the angiogenesis mediated disorder.

12) The method of claim 7, 8, 9, 10, or 11, wherein the angiogenesis modulating protein is selected from the group consisting of SEQ ID NOS. 2, 4, 6, 8, 10, 12, 14, 16, 18, 20, 22, 24, 26, 28, 30, 32, 34, 36, 38, 40, 42, 44, 46, 48-80, 82, 84, 86, 88, 90, 92, 93, 95, 97, 99, 101, 103, 150, 107, 109, 111, 113, 115-133, 135-153, 155, 157, 159, 161, 163, 165, 167, 169-172, 174, 176, 178, 180, 182, 184-185, 187, 189, 191, 193, 194, 196, 197, 199, 201, 203, 205, 207, 209-211, 213, 215, 217, 219-236, 238, 240, 242, 244-258, 260, 262, 264, 265, 267, 269, 271, 273, 275, 277, 279, 281, 283, 285, 287, 289, 291, 293, 295, 297, 298, 300, 302, 304, 306 and 308

13) The composition of claim 9, wherein the agent is selected from the group consisting of peptide, peptidomimetic, polypeptide, protein, chemical compound, nucleotide, antibody, small molecule, vitamin derivative and carbohydrate.
